(19) United States
(12) Patent Application Publication
(10) Pub. No.: US 2021/0009604 A1
(43) Pub. Date: Jan. 14, 2021

(54) BIFURAN-IMIDE-BASED MATERIALS AND USES THEREOF

(71) Applicant: YISSUM RESEARCH DEVELOPMENT COMPANY OF THE HEBREW UNIVERSITY OF JERUSALEM LTD, Jerusalem (IL)

(72) Inventors: Ori Gidron, Modi-in (IL); Sandip V. Mulay, Jerusalem (IL)

(21) Appl. No.: 16/982,914

(22) PCT Filed: May 6, 2019
(86) PCT No.: PCT/IL2019/050511

§ 371 Date: Sep. 21, 2020

Related U.S. Application Data
(60) Provisional application No. 62/667,645, filed on May 7, 2018.

(51) Int. Cl. C07D 491/153; C07D 307/68; C07D 493/14; H01L 51/00; C07D 519/00

(52) U.S. Cl. CPC C07D 491/153; C07D 307/68; C07D 493/14; H01L 51/5012; H01L 51/0065; H01L 51/0073; C07D 519/00; H01L 51/0071

(57) ABSTRACT

The invention concerns bifuran imide and methods of using same in the construction of furan-based oligomers and polymers. The furan-based systems are used for the construction of organic electronics. (formula)

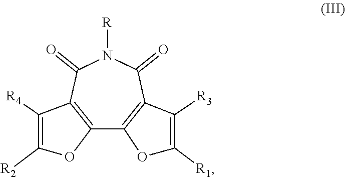

### TECHNOLOGICAL FIELD

The invention generally concerns bifuran imide and methods of using same in the construction of furan-based polymers for organic electronics.

### BACKGROUND

The field of organic electronics depends on the discovery of new materials which π-conjugated backbones endow them with desired properties. Good candidate materials are those that demonstrate small gaps between their highest occupied and lowest unoccupied molecular orbitals (small HOMO-LUMO gaps), strong fluorescence and good solid-state packing. A planar backbone results in good charge delocalization and mobility. These criteria are currently met by only a limited number of materials, with oligo- and polythiophenes and their derivatives currently dominating the field (nT, see FIG. 1).

In a previous report, a new family of organic electronic materials have been disclosed, being the oxygen-containing analogues of oligothiophenes, namely, oligofurans (nF, n=3-9, FIG. 1). Oligofurans display significant advantages over oligothiophenes, such as high rigidity/planarity, strong fluorescence, extensive conjugation and charge delocalization, good field-effect mobility, high solubility, and availability from renewable resources. These properties make oligofurans excellent candidates for the design of organic electronic materials, such as organic light emitting diodes (OLEDs), organic light emitting transistors (OLETs), organic field-effect transistor (OFETs), and organic semi-conductors. However, oligofurans commonly undergo photooxidation under ambient conditions. The resulting low environmental stability must be overcome for oligo- and polyfurans to become useful as active materials in organic electronic devices.

Bithiophene-imide (BTI, FIG. 1) was introduced by the Marks group, as an n-type semiconductor [1,2]. This building unit was successfully applied in OFETs and organic solar cells [3,4]. Ladder oligomers and polymers of BTI units were shown to have electron mobilities of up to 3.71 cm² V⁻¹ s⁻¹ and high environmental stabilities [5]. Rupar's group reported bridged bifurans complexes stabilized by p-block elements that have a lower LUMO level through σ*-π*conjugation [6].

### REFERENCES

[1] J. A. Letizia, M. R. Salata, C. M. Tribout, A. Facchetti, M. A. Ratner and T. J. Marks, J. Am. Chem. Soc., 2008, 130, 9679-9694;
[2] X. Guo, R. P. Ortiz, Y. Zheng, Y. Hu, Y.-Y. Noh, K.-J. Baeg, A. Facchetti and T. J. Marks, J. Am. Chem. Soc., 2011, 133, 1405-1418;
[3] M. Saito, I. Osaka, Y. Suda, H. Yoshida and K. Takimiya, Adv. Mater., 2016, 28, 6921-6925;
[4] Y. Wang, Z. Yan, H. Guo, M. A. Uddin, S. Ling, X. Zhou, H. Su, J. Dai, H. Y. Woo and X. Guo, Angew. Chem. Int. Ed., 2017, 56, 15304-15308;
[5] Y. Wang, H. Guo, S. Ling, I. Arrechea-Marcos, Y. Wang, J. T. L. Navarrete, R. P. Ortiz and X. Guo, Angew. Chem. Int. Ed., 2017, 56, 9924-9929;
[6] H. Cao, I. A. Brettell-Adams, F. Qu and P. A. Rupar, Organometallics, 2017, 36, 2565-2572.

### GENERAL DESCRIPTION

Herein, the inventors of the technology disclose the development of bifuran-imide units as building units for the construction of stable furan linear or cyclic oligomers and polymers (nBFI) bearing a variety of functional groups, e.g., alkyl substituents such as hexyl (nBFI-H) or 2-ocyldodecyl (nBFI-OD) substituents.

The technology disclosed herein demonstrates the significantly better stability of bifuran-imide as compared with α-oligofurans and their potential uses.

The stable furan linear or cyclic oligomers and polymers (nBFI) of the invention have been prepared from novel building blocks that may be used in the construction of even higher or larger structural homologues of the compounds disclosed and exemplified herein. Thus, the invention further provides a synthetic toolbox comprising a variety of starting materials and intermediates that may be highly useful in the synthesis of not only furan-based materials but also in a variety of other related materials.

The innovative synthetic toolbox comprises a plurality of furan derivatives that may be substituted to afford further structural modifications, thereby granting the synthetic chemist with the ability to tailor different or improved electronic, optic or physical characteristic to the final compound.

In a first aspect, there is provided a compound of the general Formula (I):

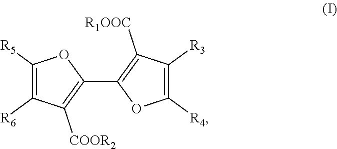

wherein

each of R₁ and R₂, independently of the other, may be selected from H, C₁-C₃₀alkyl, C₂-C₁₀alkenyl, C₂-C₁₀alkynyl, C₆-C₁₀aryl, C₅-C₁₀heteroaryl comprising between 1 and 5 heteroatoms selected from N, O, S;

each of R₃, R₄, R₅ and R₆, independently of the other, may be selected from H, C₁-C₃₀alkyl, C₂-C₁₀alkenyl, C₂-C₁₀alkynyl, C₆-C₁₀aryl, C₅-C₁₀heteroaryl comprising between 1 and 5 heteroatoms selected from N, O, S; —CN, —CO₂H, —OH, —SH, —NR′R″R″′, —NO₂, (C₁-C₃₀alkyl)₃Si— (C₁-C₃₀alkyl)₃Sn—, trifluoromethanesulfonate (triflate) and halogen, wherein each of R′, R″ and R″′ may be the same or different and may be selected from hydrogen, C₁-C₁₀alkyl, C₂-C₁₀alkenyl, C₂-C₁₀alkynyl, C₆-C₁₀aryl; wherein in case of charged amine groups, the group is associated with at least one counter-ion.

In some embodiments, at least one of R₁ and R₂ is H. In some embodiments, both of R₁ and R₂ are H.

In some embodiments, at least or both of R₁ and R₂ is an alkyl or aryl, thereby forming an ester. The alkyl or aryl may be selected from C₁-C₃₀alkyl, C₂-C₁₀alkenyl, C₂-C₁₀alkynyl, C₆-C₁₀aryl, C₅-C₁₀heteroaryl comprising between 1 and 5 heteroatoms selected from N, O, S.

In some embodiments, at least one of R₃, R₄, R₅ and R₆ is H. In some embodiments, at least two of R₃, R₄, R₅ and R₆ are H. In some embodiments, each of R₃, R₄, R₅ and R₆ is H.

In some embodiments, at least one of R₃, R₄, R₅ and R₆ is C₁-C₃₀alkyl. In some embodiments, at least two of R₃, R₄, R₅ and R₆ are C₁-C₃₀alkyl. In some embodiments, each of R₃. R₄, R and R₆ is C₁-C₃₀alkyl.

The invention further provides 2,2-bifuran-3,3′-dicarboxylic acid (herein compound 2), being a compound of Formula (I), having the structure:

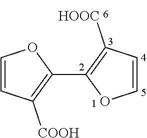 [SMILES: O=C(O)c1ccoc1-c1occc1C(=O)O]

or a derivative thereof.

The invention further provides use of compound 2 in the construction of furan based materials.

Further provided is a compound of Formula (II):

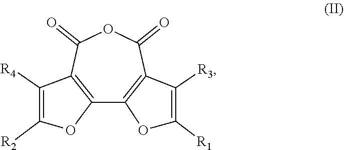

wherein

each of R₁, R₂, R₃ and R₄, independently of the other, may be selected from hydrogen, C₁-C₃₀alkyl, C₂-C₁₀alkenyl, C₂-C₁₀alkynyl, C₆-C₁₀aryl, C₅-C₁₀heteroaryl comprising between 1 and 5 heteroatoms selected from N, O, S; —CN, —CO₂H, —OH, —SH, —NR′R″R″′, —NO₂, (C₁-C₃₀alkyl)₃Si—, (C₁-C₃₀alkyl)₃Sn—, trifluoromethanesulfonate (triflate) and halogen, wherein each of R′, R″ and R″′ may be the same or different and may be selected from hydrogen, C₁-C₁₀alkyl, C₂-C₁₀alkenyl, C₂-C₁₀alkynyl, C₆-C₁₀aryl; wherein in case of charged amine groups, the group is associated with at least one counter-ion.

In some embodiments, at least one of R₁ and R₂ is H. In some embodiments, both of R₁ and R₂ are H.

In some embodiments, at least one of R₃ and R₄ is H. In some embodiments, both of R₃ and R₄ are H.

In some embodiments, at least one of R₁, R₂, R₃ and R₄ is H. In some embodiments, at least two of R₁, R₂, R₃ and R₄ are H. In some embodiments, each of R₁, R₂, R₃ and R₄ is H.

The invention further provides 2,2′-bifuran-3,3′dicarboxylic anhydride (herein compound 3), a compound of Formula (II), having the structure

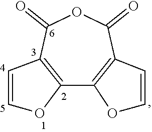 [SMILES: O=c1oc(=O)c2ccoc2c2occc12]

or a derivative thereof.

The invention further provides a compound of the general Formula (III):

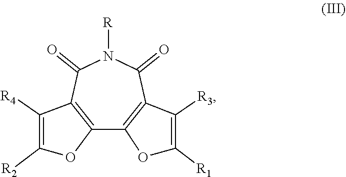

wherein

R is selected from H, C₁-C₃₀alkyl, C₂-C₁₀alkenyl, C₂-C₁₀alkynyl, C₆-C₁₀aryl, C₅-C₁₀heteroaryl comprising between 1 and 5 heteroatoms selected from N, O, S; —CN and —CO₂H;

each of R₁, R₂, R₃ and R₄, independently of the other, may be selected from hydrogen, C₁-C₃₀alkyl, C₂-C₁₀alkenyl, C₂-C₁₀alkynyl, C₆-C₁₀aryl, C₅-C₁₀heteroaryl comprising between 1 and 5 heteroatoms selected from N, O, S; —CN, —CO₂H, —OH, —SH, —NR′R″R″′, —NO₂, (C₁-C₃₀alkyl)₃Si—, (C₁-C₃₀alkyl)₃Sn—, trifluoromethanesulfonate (triflate), halogen and R_{A} (further defined hereinbelow), wherein each of R′, R″ and R″′ may be the same or different and may be selected from hydrogen, C₁-C₁₀alkyl, C₂-C₁₀alkenyl, C₂-C₁₀alkynyl, C₆-C₁₀aryl; wherein in case of charged amine groups, the group is associated with at least one counter-ion.

In some embodiments, at least one of R₁ and R₂ is H. In some embodiments, both of R₁ and R₂ are H.

In some embodiments, at least one of R₃ and R₄ is H. In some embodiments, both of R₃ and R₄ are H.

In some embodiments, at least one of R₁ and R₂ is a halide selected from Br, I, Cl, and F. In some embodiments, both of R₁ and R₂ are halides, as selected. In some embodiments, the halide is Br.

In some embodiments, at least one of R₁ and R₂ is (C₁-C₃₀alkyl)₃Sn—. In some embodiments, both of R₁ and R₂ are (C₁-C₃₀alkyl)₃Sn—.

In some embodiments, at least one of R₁, R₂, R₃ and R₄ is H. In some embodiments, at least two of R₁, R₂, R₃ and R₄ are H. In some embodiments, each of R₁, R₂, R₃ and R₄ is H.

In some embodiments, at least one of R₁ and R₂ is C₁-C₃₀alkyl. In some embodiments, both of R₁ and R₂ are C₁-C₃₀alkyl.

In some embodiments, at least one of R₁, R₂, R₃ and R₄ is C₁-C₃₀alkyl. In some embodiments, at least two of R₁, R₂, R₃ and R₄ are C₁-C₃₀alkyl.

As used herein, the group R_{A} is a group selected from:

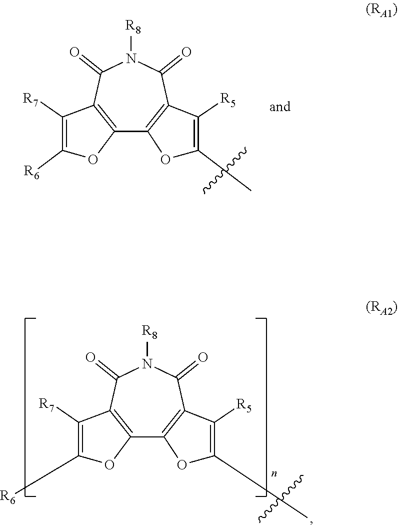

wherein

the wavy line designates a bond formed between R_{A1} and position R₁, R₂, R₃ or R₄ on a compound of Formula (III);

each of R₅, R₆, R₇ and R₈, independently of the other, may be selected from hydrogen, C₁-C₃₀alkyl, C₂-C₁₀alkenyl, C₂-C₁₀alkynyl, C₆-C₁₀aryl, C₅-C₁₀heteroaryl comprising between 1 and 5 heteroatoms selected from N, O, S; —CN, —CO₂H, —OH, —SH, —NR′R″R″′, —NO₂, (C₁-C₃₀alkyl)₃Si—, (C₁-C₃₀alkyl)₃Sn—, trifluoromethanesulfonate (triflate) and halogen, wherein each of R′, R″ and R″′ may be the same or different and may be selected from hydrogen, C₁-C₁₀alkyl, C₂-C₁₀alkenyl, C₂-C₁₀alkynyl, C₆-C₁₀aryl; wherein in case of charged amine groups, the group is associated with at least one counter-ion; or a covalent bond;

n is an integer indicating the number of repeating monomers, being between 1 and 100;

such that when R_{A} is R_{A2}, in a repeating end of a chain monomer, R₆ may be H or C₁-C₃₀alkyl; and in a repeating mid-chain monomer, R₆ is a bond connecting two monomers.

In some embodiments, R₈ is selected from H, C₁-C₃₀alkyl, C₂-C₁₀alkenyl, C₂-C₁₀alkynyl, C₆-C₁₀aryl, C₅-C₁₀heteroaryl comprising between 1 and 5 heteroatoms selected from N, O, S; —CN and —CO₂H In some embodiments, at least one of R₅, R₆, R₇ and R₈ is H. In some embodiments, both of R₆ and R₈ are H.

In some embodiments, at least two of R₅, R₆, R₇ and R₈ are H. In some embodiments, each of R₅, R₆, R₇ and R₈ is H.

In some embodiments, at least one of R₆ and R₈ is C₁-C₃₀alkyl. In some embodiments, both of R₆ and R₈ are C₁-C₃₀alkyl.

In some embodiments, at least one of R₅, R₆, R₇ and R₈ is C₁-C₃₀alkyl. In some embodiments, at least two of R₅, R₆, R₇ and R₈ are C₁-C₃₀alkyl.

In some embodiments, R₆ is Sn(C₁-C₃₀alkyl)₃ or a halide selected from Br, I, Cl and F. In some embodiments, R₆ is Br or Sn(C₁-C₃₀alkyl)₃.

In some embodiments, R₆ is Sn(C₁-C₃₀alkyl)₃ and each of R₅, R₇ and R₈ is C₁-C₃₀alkyl or H. In some embodiments, R₆ is Sn(C₁-C₃₀alkyl)₃ and each of R₅, R₇ and R₈ is H.

The invention further provides 2,2′-bifuran-3,3′-dicarboximide, a compound of Formula (III), having the structure general structure

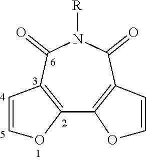

wherein R is selected from H, C₁-C₃₀alkyl, C₂-C₁₀alkenyl, C₂-C₁₀alkynyl, C₆-C₁₀aryl, C₅-C₁₀heteroaryl comprising between 1 and 5 heteroatoms selected from N, O, S; —CN and —CO₂H.

In a compound of Formula (III), the presence of a group R_{A} allows for the construction of oligomers or polymers, linear or cyclic of bifuran compounds of the invention. Thus, the invention further provides dimers, trimers, tetramers, pentamers, hexamers, heptamers, octamers, nonamers, decamers and higher homologues of a compound of Formula (III), wherein one or both of R₁ and R₂ is R_{A2}. In some embodiments, the number of furan rings in higher homologues, e.g., oligomers or polymers, is at least 10, 20, 30, 40, 50, 60, 70, 80, 90, 100, 110, 120, 130, 140, 150, 160, 170, 180, 190 or 200, or between 10 and 20, between 20 and 30, between 30 and 40, between 40 and 50, between 50 and 60, between 60 and 70, between 70 and 80, between 80 and 90, between 90 and 100, between 100 and 110, between 110 and 120, between 120 and 130, between 130 and 140, between 140 and 150, between 150 and 160, between 160 and 170, between 170 and 180, between 180 and 190, between 190 and 200.

In some embodiments, the number of furan rings in oligomers or polymers of the invention is between 3 and 50, between 3 and 45, between 3 and 40, between 3 and 35, between 3 and 30, between 3 and 25, between 3 and 20, between 3 and 15, between 3 and 10, between 3 and 9, between 3 and 8, between 3 and 7, between 3 and 6, between 3 and 5, between 5 and 45, between 5 and 40, between 5 and 35, between 5 and 30, between 5 and 25, between 5 and 20, between 5 and 15, between 5 and 10, between 10 and 45, between 10 and 40, between 10 and 35, between 10 and 30, between 10 and 25, or between 10 and 15.

In some embodiments, the number of furan rings in compounds of the invention is between 3 and 16, between 3 and 15, between 3 and 14, between 3 and 13, between 3 and 12, between 3 and 11, between 3 and 10, between 3 and 9, between 3 and 8, between 3 and 7 or between 3 and 6.

The invention further provides a compound of the general Formulae (IV) or (V) or (VI):

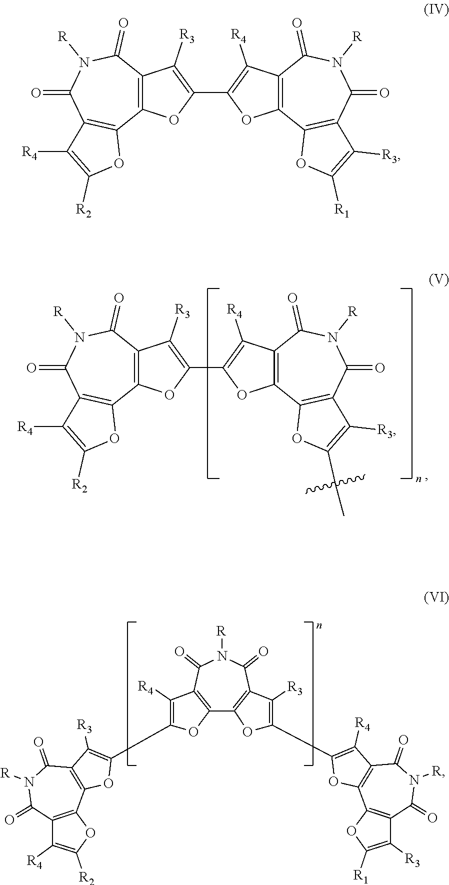

wherein

in each of compounds of Formulae (IV), (V) and (VI): each R, R₁, R₂, R₃ and R₄ may be the same or different and may be selected as indicated herein above, e.g., with reference to Formula (III). In a compound of Formula (V), the wavy line designates a point or a bond of connectivity to yield higher homologues of the indicated dimer. The integer n designates the number of monomer units, and may be selected between 1 and 30.

Also provided is the macrocycle having the oligofuran backbone, the bifuranimide α,α′-tetramer C-4BFI, shown below, wherein each R may be the same or different and may be as selected from H, C₁-C₃₀alkyl, C₂-C₁₀alkenyl, C₂-C₁₀alkynyl, C₆-C₁₀aryl, C₅-C₁₀heteroaryl comprising between 1 and 5 heteroatoms selected from N, O, S; —CN and —CO₂H.

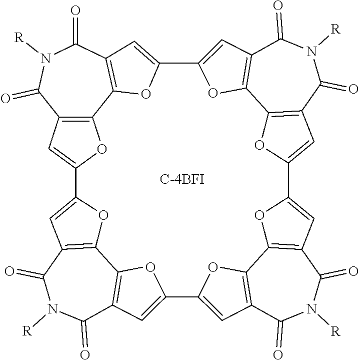

In some embodiments, compounds of the general Formula (V) or (VI) may be linear or cyclic. Where the compound is cyclic, for example, the wavy line designates bond of connectivity to the carbon atom substituted with variant R₂.

In dimers, trimers, tetramers, . . . etc, oligomers or polymers of Formula (V) or (VI), each of the variant groups (designated R, R₁, R₂ . . . etc) may be the same or different. Each variant may be different. The integer n designates the number of monomer units, and may be selected between 1 and 30.

In some embodiments, in a compound of Formula (VI), R₁ and R₂ may be H or as selected above. In some embodiments, in a compound of Formula (VI), R₁ and R₂ may be a halide selected from Br, I, Cl and F or Sn(C₁-C₃₀alkyl)₃.

As used herein with reference to any of the aforementioned specific or general Formulae structures, the variant “C₁-C₃₀alkyl” is an alkyl having between 1 and 30 carbon atoms, or between 5 and 30, 10 and 30, 15 and 30, 20 and 30, 25 and 30, 1 and 25, 1 and 20, 1 and 15, 1 and 10, 1 and 5, 2 and 30, 3 and 29, 4 and 28, 5 and 27, 6 and 26, 7 and 25, 8 and 24, 9 and 23, 10 and 22, 11 and 21, 12 and 20, 13 and 19, 14 and 18, 15 and 17, 1 and 5, 5 and 10, 10 and 15, 15 and 20 or between 20 and 30 carbon atoms. The C₁-C₃₀alkyl may be a linear alkyl or a branched alkyl group. The C₁-C₃₀alkyl may be substituted or unsubstituted.

In some embodiments, in all cases where C₁-C₃₀alkyl (e.g., as an alkyl variant or in the group Sn(C₁-C₃₀alkyl)₃, or in any other variant group) is selected, it may be selected as above, or from alkyls having between 1 and 29, 1 and 28, 1 and 27, 1 and 26, 1 and 25, 1 and 24, 1 and 23, 1 and 22, 1 and 21, 1 and 20, 1 and 19, 1 and 18, 1 and 17, 1 and 16, 1 and 15, 1 and 14, 1 and 13, 1 and 12, 1 and 11, 1 and 10, 1 and 9, 1 and 8, 1 and 7, 1 and 6, 1 and 5, 1 and 4 or between 1 and 3 carbon atoms.

In some embodiments, the C₁-C₃₀alkyl group is selected from alkyl groups having between 1 and 5, or 1 and 10, or 1 and 15, or between 1 and 20 carbon atoms.

In some embodiments, the C₁-C₃₀alkyl group is selected from alkyl groups having between 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 carbon atoms.

As used herein with reference to any of the aforementioned specific or general Formulae structures, variant R (in the endocyclic group N—R) is selected as above or from alkyls having between 1 and 29, 1 and 28, 1 and 27, 1 and 26, 1 and 25, 1 and 24, 1 and 23, 1 and 22, 1 and 21, 1 and 20, 1 and 19, 1 and 18, 1 and 17, 1 and 16, 1 and 15, 1 and 14, 1 and 13, 1 and 12, 1 and 11, 1 and 10, 1 and 9, 1 and 8, 1 and 7, 1 and 6, 1 and 5, 1 and 4 or 1 and 3. In some embodiments, R is an alkyl group selected from alkyls having between 1 and 5, or 1 and 10, or 1 and 15, or between 1 and 20 carbon atoms.

Any of the alkyl variants recited herein may be substituted or unsubstituted. In cases where the alkyl is substituted, any one or more hydrogen atom may be replaced by one or more atoms or groups of atoms. For example, an alkyl being designated as C₁-C₅alkyl comprises between 1 and 5 carbon atoms and a number of additional atoms. In cases where the alkyl is not substituted, a C₁-C₅alkyl comprises between 3 and 11 hydrogen atoms. Each of these hydrogen atoms may be relaced by a different atom or a group of atoms. Substitutions may be selected from halides such as I, Br, Cl and F, —CF₃, —OH, —NO₂, amines, —COOH or esters thereof, aryl groups and others.

In some embodiments, the alkyl is a fluorinated, e.g., perfluorinated, alkyl.

It should be noted that where the alkyl is substituted by two non-hydrogen atoms, it may be regarded an alkylene.

The invention further provides a compound selected from:

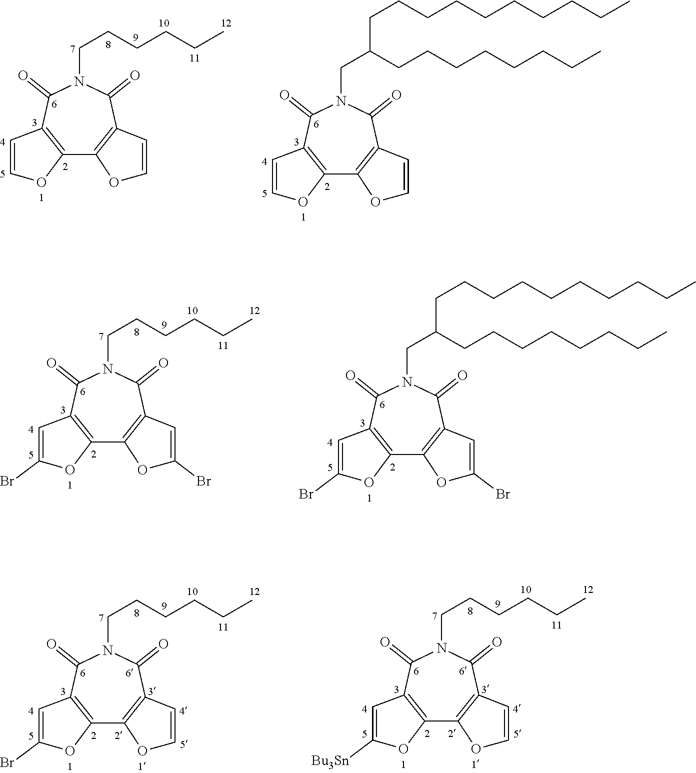

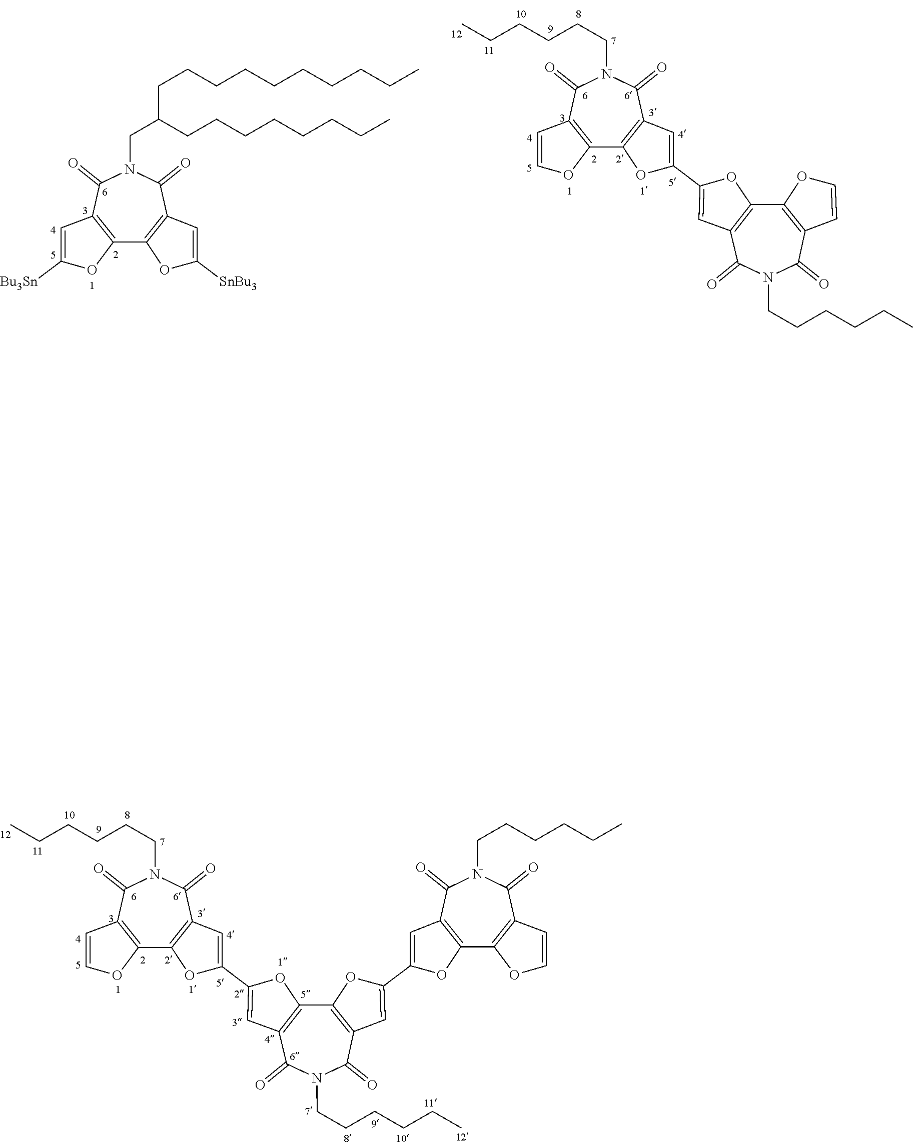

and substituted derivatives thereof.

The invention further provides a method of synthesis of a compound of the general Formula (I), the method comprising reacting a dihalobifuran under lithiation conditions in the presence of carbon dioxide to afford the diacidbifuran compound. The dihalo precursor may be selected from any dihalobifuran, e.g., dibromobifuran. The reaction may be carried out in the presence of a lithiation agent such as butyllithium, and subsequently quenched with carbon dioxide.

The invention further provides a method of synthesis of a compound of the general Formula (II), the method comprising reacting a bifuran-dicarboxylic acid under condensation/cyclization conditions in the presence of acetic anhydride.

The invention further provides a method of synthesis of a dimer, trimer, a tetramer, a higher homologue, an oligomer or a polymer of a compound of general Formula (III), (IV), (V) or (VI), the method comprising reacting a functionally substituted bifuran-imide (optionally obtained from bifuran dicarboxylic anhydride by reaction with an alkyl amine such as hexylamine or 2-octyldodecylamine, followed by in situ treatment with SOCl₂) under conditions, such as the Stille coupling conditions, to afford a dimer of the bifuran imide. The dimer may be further substituted to afford the trimer and higher homologues.

In some embodiments, functionalized bifuran imide, such a mono- or dibromobifuran imide, was reacted under lithiation conditions in the presence of a trialkyl tin halide to afford the trialkylstannyl derivative. The stannyl derivative may for example be formed by reacting the bifuran imide with a lithiation agent such as lithium diisopropylamide (LDA) followed by treatment with tributyltin chloride.

The method thus afford a bifuranimide mono or disubstituted with a trialkylstannyl group(s), e.g., tributylstannyl group(s).

The stannyl substituted bifuran imide may thereafter be reacted under the Pd-catalyzed Stille coupling reaction, attaching e.g., a monobromobifuran-imide or a dibromobifuran-imide to a stannyl derivative, to provide a dimer, a trimer or higher homologs.

In some embodiments, the bifuran imide is brominated or otherwise substituted to afford a functionalizes bifuran imide derivative that can be chain elongated or further treated to dimerize, trimerize or provide higher homologs such as oligomers and polymers. For example, bromination of bifuran imide may be achieved with molecular bromine to give monobromobifuran-imide and dibromobifuran-imide, depending on the amount of the bromine used.

Compounds of the invention thus may be used in methods of synthesis of oligomers or polymers comprising bifuranimide units. In some embodiments, compounds so obtained may comprise one or more bifuran imide units. The length of the oligomer or polymer that is based on the bifuran imide unit may be determined based on the number of furan rings (wherein each bifuran imide unit comprises two furan rings) or on the number of bifuran imide units. Notwithstanding the length of the oligomer or polymer, each bifuran imide unit in an oligomer or polymer of the invention may be the same or different. The differences in the bifuran units may be, for example, in the N-substitution, in the substitution of one furan ring as compared to the other within the same bifuran imide unit, in the substitution of one bifuran imide unit in comparison to another unit, in the positions of substitution, in the presence of one or more linker or midchain groups that are not bifuran imide units, and others.

Each oligomer or polymer of the invention may be used for a different application or for the construction of a different device. As further detailed below, compounds of the invention may be used for the construction of high molecular weight polymers, which unlike their thiophene analogues, are better candidates as p-type semiconductors. Compounds of the invention show considerable photostability, with little bleaching even after prolonged ambient light exposure, and thus are suitable building unit for organic electronics. In fact, these building units exhibit blue emission in an almost-perfect match with the European Broadcasting Union (EBU) blue standard. Combined with a good fluorescence efficiency, they can be applied in light emitting devices as blue emitters.

Thus, the invention further provides a device constructed of or comprising a blue emitter in the form of a compound according to the present invention. The device may be a blue light emitting device of any type known in the art, such as OLED and OLET.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 presents structures of nF, nT, nBFI and nBTI discussed herein.

FIG. 2 is a schematic representation of the synthetic procedure for producing bifuran-imide building units, dimer, trimer, and homopolymer. Reagents and conditions: (a) n-butyllithium, dry ice, −70° C., 7 hr; (b) Acetic anhydride, reflux, 12 hr; (c) (i) RNH₂ (R=n-hexyl or 2-octyldodecyl), CH₂Cl₂, 40° C., 12 hr, (ii) SOCl₂, 40° C., 15 hr; (d) Br₂, FeCl₃ (cat.), CH₂Cl₂, room temperature; (e) lithium diisopropylamide, tributyltin chloride, tetrahydrofuran, −85° C., 3 hr; (f) Pd(PPh₃)₄ (i.e., Pd[(C₆H₅)₃P]₄), toluene, 90° C., (g) Pd(PPh₃)₄, dimethylformamide, 90° C.

FIGS. 3A-C present ORTEP representations of (A) 3; (B) BFI-H; and (C) 2BFI-H.

FIGS. 4A-B provide packing structures of (A) BFI-H; and (B) 2BFI-H.

FIGS. 5A-D present normalized (A) UV-vis absorption and (B) fluorescence spectra of BFI-H, 2BFI-H, 3BFI-H, BFI-OD, BTI-OD, and poly(BFI-OD) in CHCl₃ at room temperature; (C) Photograph of fluorescence emission of BFI-H, 2BFI-H, 3BFI-H, and poly(BFI-OD): (top) in CHCl₃ solution and (bottom) in the solid state at 365 nm; (D) Emission colors of BFI-H, 2BFI-H, 3BFI-H, and poly(BFI-OD) on the CIE 1931 chromaticity diagram.

FIG. 6 presents photostability of solutions in 1,4-dioxane in ambient light at room temperature for 4F, BFI-H, 2BFI-H, 3BFI-H and poly(BFI-OD).

FIG. 7 presents (top) Frontier molecular orbitals of 3BFI and (bottom) calculated (B3LYP/6-31G(d)) HOMO (bottom values) and LUMO (top values) energy levels, and HOMO-LUMO gaps (middle) of the bifuran-imides discussed (in eV).

FIGS. 8A-C present the calculated (B3LYP/6-311G(d)) (A) strain energy per unit for C-nBFI; (B) HOMO-LUMO gap for L-nBFI and C-nBFI, where the black arrow indicates the tetramer; and (C) the HOMO and LUMO surfaces of C-4BFI.

FIG. 9 presents the synthesis of C-4BFI: Conditions: (a) Br₂, FeCl₃, CH₂Cl₂, rt, dark; (b) Pd(PPh₃)₄, toluene, 90° C.; (c) Ni(COD)₂, 2,2′-bipyridine, THF, 50° C., 48 h.

FIGS. 10A-D provide the X-ray structure of C-4BFI. (A) Ellipsoid representation and (B) packing in stick representation excluding the 2-octyldodecyl groups. Hydrogens and solvent molecules are omitted for clarity. (C) Electrostatic potential map of C-4BFI dimer, calculated at the B3LYP/6-311G(d) level. (D) Stick representation of C-4BFI column, including 2-octyldodecyl side groups, showing slip-stack packing arrangement.

FIG. 11 provides normalized UV-Vis absorption (solid line) and emission (dashed line) spectra for L-4BFI, C-4BFI in chloroform, and C-4BFI film. Inset: photograph of solid C-4BFI fluorescing upon illumination with UV light (365 nm).

FIGS. 12A-D provide: (A) STM image of C-4BFI (5×10⁻² M) at the 1,2,4-trichlorobenzene-graphite interface. Image scale: 19.3×19.3 nm². Imaging conditions: Iₛₑₜ=1 pA, and V_{bias}=−1.2 V. (B) Molecular modelling unit cell parameters: a=1.7±0.1 nm, b=2.4±0.1 nm and γ=84±1°. (C) STM image showing the multilayer. Image scale: 13.6×13.6 nm². Blue arrow shows the vacancy defect in the upper layer through which the STM contrast still shows a molecular structure (from the layer below). (D) Molecular model displaying C═O . . . H hydrogen bonding between two adjacent macrocycles.

FIG. 13 provides a synthetic scheme for the preparation of C-4BFI.

### DETAILED DESCRIPTION OF EMBODIMENTS

Reagents and chemicals used here are commercially available and were used without further purification unless otherwise stated. Flash chromatography (FC) was performed using CombiFlash SiO₂ columns. The compounds 3,3′-dibromo-2,2′-bifuran, 2-octyldodecylamine, and N-(2-octyldodecyl)-2,2′-bithiophene-3,3′dicarboximide were synthesized according to the procedures detailed in the literature.

¹H and ¹³C NMR spectra were recorded in solution on a Brücker-AVIII 400 MI-z and 500 MHz spectrometer using tetranethylsilane (TMS) as the external standard. The spectra were recorded using chloroform-d, methanol-d4 as solvents. Chemical shifts are expressed in δ units. High resolution mass spectra were measured on a HR Q-TOF LCMS and Waters Micromass GCT_Preniier Mass Spectrometer using ESI and field desorption (FD) ionization. Molecular weights and size distribution were determined by gel permeation chromatography (GPC) in chloroform (with 0.025% toluene as internal standard) against PS standards with a flow rate of 1 ml/min. GPC was performed on a Polymer Standards Service (PSS) system consisting of a PSS SDV linear M column, refractive index and UV detectors (Thermo). Data analysis and standard calibration (with PS standards) were performed using PSS WinGPC UniChrom software V8.20. Build 5350. UV-vis absorption and fluorescence spectra of synthesized compounds were recorded on an Agilent Technologies Cary series UV-Vis-NIR spectrophotometer. Fluorescence measurements were carried out with a Horiba Scientific Fluoromax-4 spectrofluorometer.

### 3,3′Dibromo-2,2′-bifuran (1)

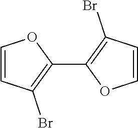 [SMILES: Brc1ccoc1-c1occc1Br]

A solution of lithium diisopropylamide (LDA) (37.4 mL, 2.0 M in hexane) was added dropwise to a solution of 3-bromo-furan (10 g, 68.04 mmol) in dry THF (60 mL) at −78° C. under N₂. The reaction mixture was then stirred under the same conditions. After 1.5 h, CuCl₂ (10 g, 74.8 mmol) was added in one portion and the resulting solution was allowed to reach room temperature slowly and was stirred overnight. The reaction mixture was then added into 100 ml water with 5 g glycine at 0° C., filtered, filtrate extracted with diethyl ether (3×80 mL), dried (Na₂SO₄) and concentrated. The residue obtained was purified by flash column chromatography on silica gel with hexane as eluent to give 1 as a white solid (6.7 g, 69%). ¹H NMR (400 MHz, Chloroform-d): δ 7.47 (d, J=1.9 Hz, 2H), 6.55 (d, J=1.9 Hz, 2H).

### 2,2′-Bifuran-3,3′-dicarboxylic Acid (2)

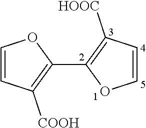 [SMILES: O=C(O)c1ccoc1-c1occc1C(=O)O]

To a solution of n-BuLi (31.2 mL, 50.0 mmol, 1.6 M in hexane) in dry THF (50 mL), 3,3′-dibromobifuran (1) (5.84 g, 20.0 mmol) in dry THF (40 mL) was added dropwise over the period of 30 minutes at −70° C. The resulting solution was stirred at −70° C. until it becomes white slurry (about 2 hours). To the slurry was added dry ice (7.0 g, 160.0 mmol) at −70° C. Then, the mixture was gradually warmed to room temperature. After stirring for 5 hours at room temperature, the reaction mixture was quenched with methanol (5 mL), and the solvent was removed under a reduce pressure. The crude product was purified by flash column chromatography on silica gel using AcOH/CH₂Cl₂/methanol (1:4:95) as eluent to give 2 light yellow solid (3.38 g, 76%). ¹H NMR (400 MHz, Methanol-d): δ 7.68 (d, J=2.0 Hz, 2H; H—C (5)), 6.87 (d, J=1.9 Hz, 2H, H—C (4)); ¹³C NMR (101 MHz, Methanol-d): δ 165.75 (2C (6)), 147.34 (2C (2)), 145.11 (2C (5)), 121.08 (2C (3)), 112.99 (2C (4)); HRMS (ESI): calcd for C₁₀H₆O₆ (M+H)⁺: 223.0243, found: 223.0237.

### 2,2′-Bifuran-3,3′dicarboxylic anhydride (3)

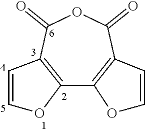 [SMILES: O=c1oc(=O)c2ccoc2c2occc12]

A solution of (2,2-bifuran)-3,3′dicarboxylic acid (2) (3.3 g, 14.9 mmol) in acetic anhydride (100 mL) was stirred under refluxed condition for 12 h. Acetic anhydride was removed by distillation under reduced pressure. The residue thus obtained was purified by flash column chromatography on silica gel using hexane/ethyl acetate (4:1) as eluent to give anhydride 3 as a light yellow solid (2.46 g, 81%). ¹H NMR (400 MHz, Chloroform-d): δ 7.61 (d, J=1.6 Hz, 2H; H—C (5)), 7.11 (d, J=1.6 Hz, 2H; H—C (4)); ¹³C NMR (101 MHz, Chloroform-d): δ 154.35 (2C (6)), 145.35 (2C (2)), 144.74 (2C (5)), 116.27 (2C (3)), 113.93 (2C (4)); HRMS (ESI): calcd for C₁₀H₆O₆ (M+H)⁺: 205.0137, found: 205.0130.

### N-Hexyl-2,2′-bifuran-3,3′-dicarboximide (BFI-H)

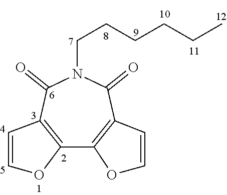 [SMILES: CCCCCCn1c(=O)c2ccoc2c2occc2c1=O]

To a solution of anhydride 3 (2.04 g, 10.0 mmol) in dry CH₂Cl₂ (100 mL) was added a solution of 1-hexylamine (1.6 mL, 12.0 mmol) in CH₂Cl₂(20 mL) dropwise under N₂. After addition, the reaction mixture was stirred under reflux 12 h. The reaction mixture was cooled to room temperature and then SOCl₂ (1.1 mL, 15.0 mmol) was added dropwise. The reaction mixture was again refluxed for 15 h. The reaction was quenched sat. aq. solution of NaHCO₃ (50 mL). The separated aqueous layer extracted with CH₂Cl₂ (2×50 mL), dried (Na₂SO₄), filtered, and concentrated. The residues obtained was purified by flash column chromatography on silica gel using hexane and ethyl acetate (4:1) as eluent to give BFI-H as a white solid (2.53 g, 88%). ¹H NMR (500 MHz, Chloroform-d): δ 7.54 (d, J=1.9 Hz, 2H; H—C (5)), 7.15 (d, J=1.9 Hz, 2H; H—C (4)), 4.21 (t, J=7.8, 2H; —CH₂(7)), 1.69-1.63 (m, 2H; —CH₂(8)), 1.42-1.30 (m, 6H; —CH₂(9-11), 0.88 (t, J=7.1 Hz, 3H; —CH₃(12)); ¹³C NMR (101 MHz, Chloroform-d): δ 160.42 (2C (6)), 143.64 (2C (5)), 143.49 (2C (2)), 120.40 (2C (3)), 114.17 (2C (4)), 45.29 (C (7)), 31.65 (C (10)), 27.61 (C (8)), 26.98 (C (9)), 22.71 (C (11)), 14.17 (C (12)); HRMS (ESI): calcd for C₁₆H₁₈NO₄ (M+H)⁺: 288.1230, found: 288.1254.

### N-Hexyl-5-bromo-2,2′-bifuran-3,3′-dicarboximide (4a)

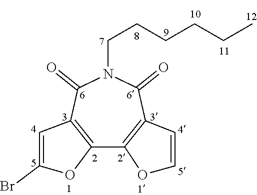 [SMILES: CCCCCCn1c(=O)c2ccoc2c2oc(Br)cc2c1=O]

Bromine (0.18 mL, 3.6 mmol) and catalytic amount of FeCl₃ (10 mg, 2 mol %) were added to the solution of imide BFI-H (0.862 g, 3.0 mmol) in CH₂Cl₂ (40 mL) and the reaction mixture was stirred in dark for 4 h. After completion of reaction it was quenched by sat. aq. solution of Na₂SO₃ and stirred for further 30 min. The reaction mixture poured into CH₂Cl₂ (150 mL) and washed with water (2×100 mL), brine, dried (Na₂SO₄), filtered, and concentrated. The residue obtained was purified by flash column chromatography on silica gel using hexane and CH₂Cl₂ (1:1) as eluent to give 4a as a white solid (0.62 g, 56%). ¹H NMR (500 MHz, Chloroform-d): δ 7.55 (d, J=1.9 Hz, 1H; H—C (5′)), 7.15 (d, J=1.9 Hz, 1H; H—C (4′)), 7.07 (s, 1H; H—C (4)), 4.18 (t, J=7.8, 2H; —CH₂(7)), 1.67-1.61 (m, 2H; —CH₂(8)), 1.40-1.30 (m, 6H; —CH₂(9-11)), 0.88 (t, J=7.1 Hz, 3H; —CH₃(12)); ¹³C NMR (125 MHz, Chloroform-d): δ 160.1 (C (6)), 159.4 (C (6′)), 144.6 (C (2′)), 144.0 (C (5′)), 142.5 (C (2)), 125.7 (C (3)), 121.9 (C (5)), 120.5 (C (3′)), 115.6 (C (4)), 114.3 (C (4′)), 45.4 (C (7)), 31.7 (C (10)), 27.6 (C (8)), 27.0 (C (9)), 22.7 (C (11)), 14.2 (C (12)); HRMS (ESI): calcd for C₁₆H₁₆Br₂NO₄ (M+H)⁺: 366.0341, found: 366.0357.

### N-Hexyl-5,5′-dibromo-2,2′-bifuran-3,3′-dicarboximide (4b)

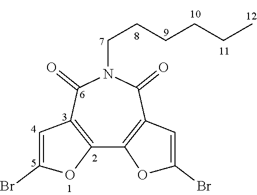 [SMILES: CCCCCCn1c(=O)c2cc(Br)oc2c2oc(Br)cc2c1=O]

Bromine (0.62 mL, 12.0 mmol) and catalytic amount of FeCl₃ (10 mg, 2 mol %) were added to the solution of imide BFI-H (0.862 g, 3.0 mmol) in CH₂Cl₂ (40 mL) and the reaction mixture was stirred in dark for 4 h. After completion of reaction it was quenched by sat. aq. solution of Na₂SO₃ and stirred for further 30 min. The reaction mixture poured into CH₂Cl₂ (150 mL) and washed with water (2×100 mL), brine, dried (Na₂SO₄), filtered, and concentrated. The residues obtained was purified by flash column chromatography on silica gel using hexane and ethyl acetate (9:1) as eluent to give 4b as a white solid (1.29 g, 97%). ¹H NMR (500 MHz, Chloroform-d): δ 7.06 (s, 2H; H—C (4), 4.16 (t, J=7.8, 2H; —CH₂(7)), 1.65-1.59 (m 2H; —CH₂(8)), 1.38-1.29 (m 6H; —CH₂(9-11)), 0.88 (t, J=7.1 Hz, 3H; —CH₃(12)); ¹³C NMR (125 MHz, Chloroform-d): δ 159.1 (2C (6)), 143.5 (2C (2)), 126.1 (2C (3)), 121.9 (2C (5)), 115.7 (2C (4)), 45.5 (C (7)), 31.6 (C (10)), 27.5 (C (8)), 26.9 (C (9)), 22.7 (C (11)), 14.2 (C (12)); HRMS (ESI): calcd for C₁₆H₁₆Br₂NO₄ (M+H)⁺: 445.9421, found: 445.9435.

### N-Hexyl-5-(tributylstannyl)-2,2′-bifuran-3,3′-dicarboximide(5)

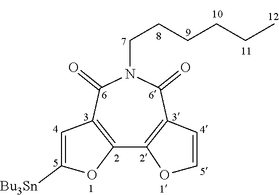

A solution of LDA (0.55 mL, 2.0 M in hexane, 1.1 mmol) was added dropwise to a solution of N-hexyl-2,2′-bifuran-3,3′-dicarboximide (BFI-H) (287 mg, 1.0 mmol) in dry THF (15 mL) at −85° C. under N₂ and stirred for 1 h. Bu₃SnCl (0.33 mL, 1.2 mmol) was added dropwise, and the reaction mixture was allowed to reach room temperature and stirred for 2 h. The mixture was quenched with water, extracted with CH₂Cl₂ (2×40 mL), dried (Na₂SO₄), filtered, and evaporated. The residue obtained was purified by flash column chromatography on silica gel basified with trimethylamine using hexane as eluent to give 5 as a colorless oil (300 mg. 52%). ¹H NMR (400 MHz, Chloroform-d): δ 7.53 (d, J=1.9 Hz, 1H), 7.25 (s, 1H), 7.15 (d, J=1.9 Hz, 1H), 4.22 (t, J=7.7, 2H), 1.68-1.55 (m, 8H), 1.39-1.30 (m, 12H), 1.20-1.15 (m, 6H), 0.90 (t, J=7.2 Hz, 12H); ¹³C NMR (101 MHz, Chloroform-d): δ 165.72, 160.90, 160.65, 147.59, 144.09, 143.22, 125.17, 120.64, 119.33, 114.05, 45.19, 31.67, 28.97 (3C), 27.67, 27.25 (3C), 27.01 22.72, 14.18, 13.74 (3C), 10.59 (3C); HRMS (ESI) calcd for C₂₈H₄₄NO₄Sn (M+H)⁺: 578.2292, found: 578.2290.

### Bis (N-hexyl-2,2′-bifuran-3,3′-dicarboximide) (2BFI-H)

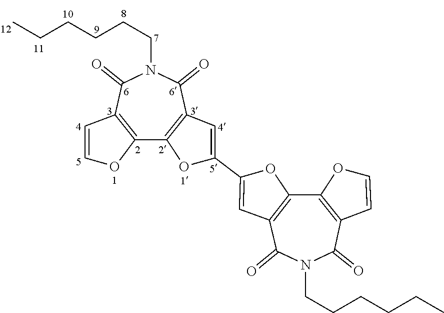 [SMILES: CCCCCCn1c(=O)c2ccoc2c2oc(-c3cc4c(=O)n(CCCCCC)c(=O)c5ccoc5c4o3)cc2c1=O]

Pd(PPh₃)₄ (18 mg, 0.016 mmol) was added to a solution of N-Hexyl-5-bromo-2,2′-bifuran-3,3′-dicarboximide (4a) (146 mg, 0.40 mmol) and N-Hexyl-5-(tributylstannyl)-2,2′-bifuran-3,3′-dicarboximide (5) (300 mg, 0.52 mmol) in dry and degassed toluene (10 mL), and the reaction mixture was stirred at 90° C. for 24 h under nitrogen. Then reaction mixture was cooled to room temperature and solvent was evaporated under reduced pressure. The residue obtained was purified by flash column chromatography on silica gel using hexane and dichloromethane (1:8) as eluent to give 2BFI-H as a yellow solid (140 mg, 61%). ¹H NMR (500 MHz, Chloroform-d): δ 7.60 (d, J=1.9 Hz, 2H; H—C (5)), 7.47 (s, 2H; H—C (4′)), 7.18 (d, J=1.9 Hz; H—C (4)), 4.21 (t, J=7.8, 4H; —CH₂(7)), 1.71-1.63 (m, 4H; —CH₂(8)), 1.42-1.30 (m, 12H; —CH₂(9-11)), 0.89 (t, J=7.1 Hz, 6H; —CH₃(12)); ¹³C NMR (125 MHz, Chloroform-d): δ 160.06 (2C (6)), 159.80 (2C (6′)), 144.78 (2C (5′)), 144.26 (2C (5)), 143.30 (2C (2′)), 142.62 (2C (2)), 121.78 (2C (3)), 121.40 (2C (3′)), 114.54 (2C (4)), 111.44 (2C (4′)), 45.42 (2C (7)), 31.66 (2C (10)), 27.60 (2C (8)), 26.98 (2C (9)), 22.73 (2C (11)), 14.19 (2C (12)); HRMS (ESI): calcd for C₃₂H₃₃N₂O₈ (M+H)⁺: 573.2237, found: 573.2229.

### Tris (N-Hexyl-2,2′-bifuran-3,3′-dicarboximide) (3BFI-H)

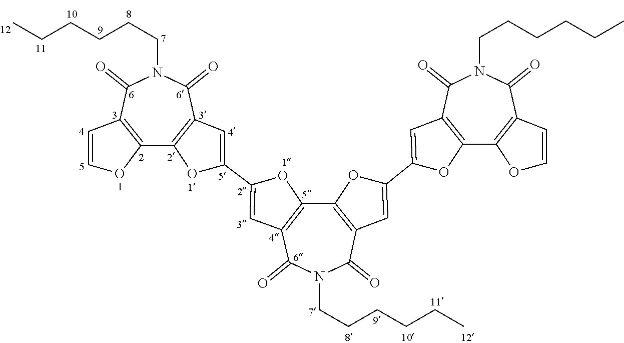 [SMILES: CCCCCCn1c(=O)c2ccoc2c2oc(-c3cc4c(=O)n(CCCCCC)c(=O)c5cc(-c6cc7c(=O)n(CCCCCC)c(=O)c8ccoc8c7o6)oc5c4o3)cc2c1=O]

Pd(PPh₃)₄ (18 mg, 0.016 mmol) was added to a solution of N-Hexyl-5,5′-dibromo-2,2′-bifuran-3,3′-dicarboximide (4b) (111 mg, 0.25 mmol) and N-Hexyl-5-(tributylstannyl)-2,2′-bifuran-3,3′-dicarboximide (5) (432 mg, 0.75 mmol) in dry and degassed toluene (10 mL), and the reaction mixture was stirred at 90° C. for 24 h under nitrogen. Then reaction mixture was cooled to room temperature and solvent was evaporated under reduced pressure. The residue obtained was purified by flash column chromatography on silica gel using dichloromethane and ethyl acetate (95:5) as eluent to give 3BFI-H as a yellow solid (96 mg, 45%). ¹H NMR (500 MHz, Chloroform-d): δ 7.63 (d, J=1.9 Hz, 2H; H—C (5)), 7.52 (s, 2H; H—C (3″)), 7.45 (s, 2H; H—C (4′)), 7.18 (d, J=1.8 Hz, 2H; H—C (4)), 4.20 (t, J=7.6, 6H; —CH₂(7)), 1.67 (quint, J=7.6 Hz, 6H; —CH₂(8)), 1.43-1.33 (m, 18H; —CH₂(9-11)), 0.90 (t, J=7.1 Hz, 3H; —CH₃(12)); ¹³C NMR (125 MHz, Chloroform-d): δ 160.01 (2C (6)), 159.68 (2C (6′)), 159.41 (2C (6″)), 145.28 (2 C (2′)), 144.49 (2C (2″)), 144.39 (2C (5)), 143.41 (2C (5″)), 142.52 (2C (2)), 142.28 (2 C (5′)), 122.72 (2C (4″)), 121.81 (2C (3)), 121.56 (2C (3′)), 114.59 (2C (4)), 112.00 (2 C (3″)), 111.66 (2C (4′)), 45.56 (1C (7′)), 45.43 (2C (7)), 31.66 (3C (10)), 27.61 (2C (8)), 27.59 (1C (8′)), 26.99 (3C (9)), 22.75 (3C (11)), 14.21 (3C (12)); HRMS (ESI): calcd for C₁₆H₁₆Br₂NO₄ (M+H)⁺: 366.03410, found: 366.03574.

### N-(2-octyldodecyl)-2,2′-bifuran-3,3′-dicarboximide (BFI-OD)

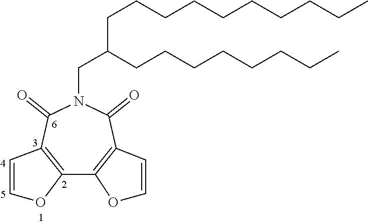 [SMILES: CCCCCCCCCCC(CCCCCCCC)Cn1c(=O)c2ccoc2c2occc2c1=O]

To a solution of anhydride 3 (1.5 g, 7.34 mmol) in dry CH₂Cl₂ (80 mL) was added a solution of 2-octyldodecylamine (2.4 g, 8.07 mmol) in CH₂Cl₂(20 mL) dropwise under N₂. After addition, the reaction mixture was stirred under reflux 12 hr. The reaction mixture was cooled to room temperature and then SOCl₂ (0.87 mL, 12.0 mmol) was added dropwise. The reaction mixture was again refluxed for 15 hr. The reaction was quenched sat. aq. solution of NaHCO₃ (20 mL). The aqueous layer extracted with CH₂Cl₂ (2×80 mL), dried (Na₂SO₄) and concentrated. The residues obtained was purified by flash column chromatography on silica gel using hexane and ethyl acetate (9:1) as eluent to give BFI-OD as a colorless oil (2.81 g, 79%). ¹H NMR (500 MHz, Chloroform-d): δ 7.54 (d, J=1.9, 2H; H—C (5)), 7.16 (d, J=1.9, 2H; H—C (4)), 4.24 (d, J=7.2, 2H; —CH₂—CH₃), 1.84 (m, 1H; —CH₂—CH₃), 1.38-1.18 (m, 32H; 16(CH₂)), 0.87 (t, J=7.1 Hz, 3H; (CH₃)), 0.86 (t, J=6.8 Hz, 3H; (CH₃)); ¹³C NMR (125 MHz, Chloroform-d): δ 160.84 (2C (6)), 143.60 (2C (5)), 143.41 (2C (2)), 120.41 (2C (3)), 114.35 (2C (4)), 48.56, 36.45, 32.07, 32.03, 31.85 (2C), 30.21 (2C), 29.79, 29.77, 29.74, 29.69, 29.48, 29.44, 26.65 (2C), 22.82, 22.80, 14.25 (2C); HRMS (ESI): calcd for C₁₆H₁₆Br₂NO₄ (M+H)⁺: 445.94211, found: 445.94357.

### N-(2-octyldodecyl)-5,5′-dibromo-2,2′-bifuran-3,3′-dicarboximide(4c)

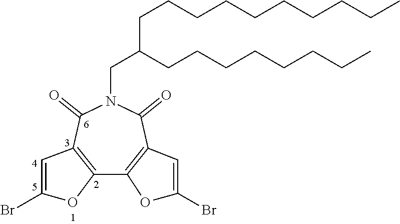 [SMILES: CCCCCCCCCCC(CCCCCCCC)Cn1c(=O)c2cc(Br)oc2c2oc(Br)cc2c1=O]

Bromine (0.32 mL, 6.0 mmol) and catalytic amount of FeCl₃ (5 mg, 2 mol %) were added to the solution of N-(2-octyldodecyl)-2,2′-bifuran-3,3′-dicarboximide (BFI-OD) (0.73 g, 3.0 mmol) in CH₂Cl₂ (40 mL) and the reaction mixture was stirred in dark for 4 hr. After completion of reaction it was quenched by sat. aq. solution of Na₂SO₃ and stirred for further 30 min. The reaction mixture poured into CH₂Cl₂ (100 mL) and washed with water (2×60 mL), brine, dried (Na₂SO₄), filtered and concentrated. The residues obtained was purified by flash column chromatography on silica gel using hexane and ethyl acetate (9:1) as eluent to give 4c as a colorless oil (0.93 g, 96%). ¹H NMR (500 MHz, Chloroform-d): δ 7.07 (s, 2H; H—C (4)), 4.19 (d, J=7.3, 2H; —CH₂—CH₃), 1.84 (m, 1H; —CH₂—CH), 1.34-1.18 (m, 32H; 16(CH₂)), 0.87 (t, J=7.0 Hz, 3H; (CH₃)), 0.86 (t, J=7.0 Hz, 3H; (CH₃)); ¹³C NMR (125 MHz, Chloroform-d) δ 159.46 (2C (6)), 143.45 (2C (2)), 126.08 (2C (3)), 121.92 (2C (5)), 115.86 (2C (4)), 48.76, 36.37, 32.08, 32.06, 31.80 (2C), 30.18 (2C), 29.80 (2C), 29.74, 29.70, 29.50, 29.46, 26.60 (2C), 22.84, 22.83, 14.27 (2C); HRMS (ESI): calcd for C₁₆H₁₆Br₂NO₄ (M+H)⁺: 445.94211, found: 445.94357.

### N-(2-octyldodecyl)-5,5′-bis(tributylstannyl)-2,2′-bifuran-3,3′-dicarboximide (6)

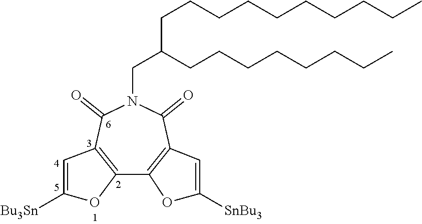

A solution of LDA (0.42 mL, 2.0 M in hexane, 0.84 mmol) was added dropwise to a solution of N-(2-octyldodecyl)-2,2′-bifuran-3,3′-dicarboximide (BFI-OD) (185 mg, 0.38 mmol) in dry THF (15 mL) at −85° C. under N₂ and stirred for 1 h. Bu₃SnCl (0.23 mL, 0.84 mmol) was added dropwise, and the reaction mixture was allowed to reach room temperature and stirred for 2 h. The mixture was quenched with water, extracted with CH₂Cl₂ (2×40 mL), dried (Na₂SO₄), filtered, and evaporated. The residue obtained was purified by flash column chromatography on silica gel basified with trimethylamine using hexane as eluent to give 6 as colorless oil (331 mg, 81%). ¹H NMR (400 MHz, Chloroform-d): δ 7.25 (s, 2H; H—C (4)), 4.25 (d, J=7.1, 2H; —CH₂—CH), 1.93-1.86 (m, 1H; —CH₂—CH), 1.64-1.56 (m, 12H; 6(CH₂) 1.42-1.13 (m, 56H; 28(CH₂)), 0.91 (t, J=7.0 Hz, 18H; (CH₃)), 0.87 (t, J=7.0 Hz, 3H; (CH₃)), 0.86 (t, J=7.0 Hz, 3H; (CH₃)); ¹³C NMR (125 MHz, Chloroform-d) δ 164.87 (2C), 161.55 (2C), 148.06 (2C), 125.30 (2C), 119.45 (2C), 48.45, 36.62, 32.08, 32.06, 31.93 (2C), 30.26 (2C), 29.81, 29.80 (2C), 29.73, 29.50, 29.46, 29.01 (6C), 27. 30 (6C), 26.77 (2C), 22.84, 22.82, 14.27 (2C), 13.79 (6C), 10.62 (6C); ); HRMS (ESI) calcd for C₅₄H₉₇NO₄Sn₂ (M)⁺: 1062.5534, found: 1062.5573.

### Poly-N-(2-octyldodecyl)-2,2′-bifuran-3,3′-dicarboximide (poly(BFI-OD))

Pd(PPh₃)₄ (29 mg, 5 mol %) was added to a solution of N-(2-octyldodecyl)-5,5′-dibromo-2,2′-bifuran-3,3′-dicarboximide (4c) (321 mg, 0.50 mmol) and N-(2-octyldodecyl)-5,5′-bis(tributylstannyl)-2,2′-bifuran-3,3′-dicarboximide (6)(531 mg, 0.50 mmol) in dry and degassed DMF (10 mL), and the reaction mixture was stirred at 90° C. for 24 h under nitrogen. The reaction mixture was then allowed to cool to room temperature and poured into 200 mL of methanol, and the precipitate was collected by filtration to give 530 mg of orange solid. The crude material was transferred to a Soxhlet extractor and extracted for 48 h with methanol and a further 24 h with acetone, and then recovered by extraction with hexane. The hexane solution was concentrated, and the polymer precipitated with 150 mL of methanol, filtered, and dried to give poly(BFI-OD) as an orange solid (350 mg 36%). ¹H NMR (500 MHz, Chloroform-d): δ 7.5 (bs, 2H), 4.20 (bs, 2H), 1.80 (bs, 1H), 1.64-1.00 (m, 32H), 0.75 (bs, 6H); ¹³C NMR (125 MHz, Chloroform-d) δ 159.19, 144.98, 141.82, 122.94, 112.04, 48.82, 36.52, 32.10, 30.31, 30.16, 29.94, 29.86, 29.82, 29.76, 29.58, 29.55, 26.77, 22.87, 14.29, 14.26; GPC (30° C.) Molecular weight: Mn=12.4 kDa, Mw=33.7 kDa, PDI=2.71.

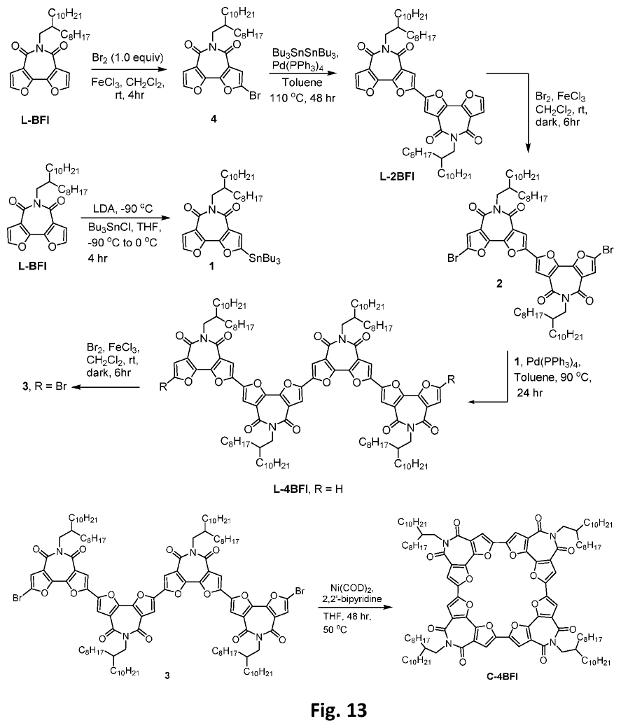

### N-(2-octyldodecyl)-5,-bromo-2,2′-bifuran-3,3′-dicarboximide (FIG. 13)

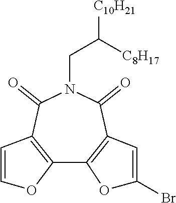

Bromine (0.21 mL, 4.13 mmol) and a catalytic amount of FeCl₃ (13 mg, 2 mol %) were added to a solution of imide L-1BFI (2.0 g, 4.13 mmol) in CH₂Cl₂ (50 mL) and the reaction mixture was stirred in the dark for 4 h. After completion of the reaction, it was quenched using a sat. aq. solution of Na₂S₂O₃ and stirred for a further 30 min. The reaction mixture was poured into CH₂Cl₂ (150 mL) and washed with water (2×100 mL), brine, dried (Na₂SO₄), filtered, and concentrated. The residue obtained was purified by flash column chromatography on silica gel using hexane and CH₂Cl₂ (1:1) as eluent to give 4a (FIG. 13) as a light yellow solid (1.71 g, 74%), ¹H NMR (500 MHz, Chloroform-d) δ 7.53 (d, J=1.9 Hz, 1H), 7.13 (d, J=1.9 Hz, 1H), 7.04 (s, 1H), 4.19 (d, J=7.2 Hz, 2H), 1.87-1.82 (m, 1H), 1.35-1.16 (m, 32H), 0.85 (t, J=7.0 Hz, 3H), 0.84 (t, J=7.0 Hz, 3H); ¹³C NMR (126 MHz, Chloroform-d) δ 160.38, 159.67, 144.43, 143.85, 142.29, 125.58, 121.87, 120.40, 115.71, 114.41, 48.58, 36.35, 32.04, 32.01, 31.78, 30.16, 29.77, 29.75, 29.71, 29.66, 29.46, 29.42, 26.58, 22.80, 22.78, 14.22; HRMS (ESI) calcd. for C₃₀H₄₅BrNO₄ 564.2511, found 564.2503 (M+H)⁺.

### N-(2-octyldodecyl)-(tributylstannyl)-2,2′-bifuran-3,3′-dicarboximide (FIG. 13)

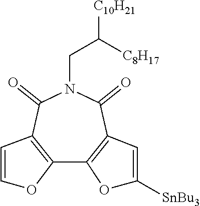

A solution of lithium diisopropylamide (LDA; 2.3 mL, 2.0 M in hexane, 4.54 mmol) was added dropwise to a solution of N-(2-octyldodecyl)-2,2′-bifuran-3,3′-dicarboximide (L-1BFI) (2.0 g, 4.13 mmol) in dry THF (80 mL) at −90° C. under N₂ and stirred for 1 hr. Bu₃SnCl (1.68 mL, 6.19 mmol) was added dropwise, and the reaction mixture was allowed to reach at 0° C. and stirred for 2 hr. The mixture was quenched with water, extracted with CH₂Cl₂ (2×80 mL), dried (Na₂SO₄), filtered, and evaporated. The residue obtained was purified by flash column chromatography on silica gel basified with trimethylamine using hexane as eluent to give 2 (FIG. 13) as a colorless oil (1.75 g, 55%). ¹H NMR (500 MHz, Chloroform-d) δ 7.53 (d, J=1.9 Hz, 1H), 7.26 (d, J=2.4 Hz, 1H), 7.15 (d, J=1.9 Hz, 1H), 4.25 (d, J=7.3 Hz, 2H), 1.89 (p, J=5.6, 5.1 Hz, 1H), 1.64-1.55 (m, 6H), 1.45-1.12 (m, 44H), 0.94-0.81 (m, 15H); ¹³C NMR (126 MHz, Chloroform-d) δ 165.69, 161.34, 161.10, 147.52, 144.02, 143.20, 125.35, 120.66, 119.35, 114.24, 48.52, 36.54, 32.07, 32.05, 31.92, 31.90, 30.25, 30.23, 29.80, 29.78, 29.77, 29.72, 29.49, 29.46, 29.00, 27.29, 26.71, 22.84, 22.82, 14.26, 13.77, 10.61; HRMS (ESI) calcd for C₄₂H₇₂NO₄Sn 774.4487, found 774.4512 (M+H)⁺.

### Bis (N-2-octyldodecyl-2,2′-bifuran-3,3′-dicarboximide) (FIG. 13, L-2BFI)

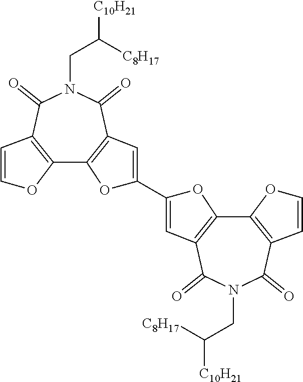

To a two-neck flask equipped with a condenser was added 4 (1.88 g, 3.34 mmol), hexabutyldistannane (969 mg, 1.67 mmol), tetrakis(triphenylphosphine)palladium(0) [Pd(PPh₃)₄] (192 mg, 0.167 mmol), and 70 mL dry toluene. The reaction mixture was refluxed under argon for 48 h. After cooling to room temperature, the solvent was evaporated under reduced pressure and the residue obtained was purified by flash column chromatography on silica gel using hexane and CH₂Cl₂ (1:3) as the eluent to give L-2BFI (FIG. 13) as a yellow solid (1.2 gm, 75%). ¹H NMR (500 MHz, Chloroform-d) δ 7.60 (d, J=1.9 Hz, 2H), 7.48 (s, 2H), 7.18 (d, J=1.9 Hz, 2H), 4.24 (d, J=7.2 Hz, 4H), 1.88 (p, J=6.1 Hz, 2H), 1.38-1.16 (m, 64H), 0.85 (t, J=7.0 Hz, 6H), 0.84 (t, J=7.0 Hz, 6H); ¹³C NMR (126 MHz, Chloroform-d) δ 160.42, 160.19, 144.79, 144.19, 143.19, 142.53, 121.78, 121.38, 114.69, 111.59, 48.63, 36.43, 32.05, 32.04, 31.84, 30.20, 29.80, 29.78, 29.75, 29.70, 29.48, 29.45, 26.63, 22.81, 14.24. HRMS (MALDI) calcd for C₆₀H₈₈N₂O₈ 964.654, found 964.668 (M)⁺.

### Compound 2 (FIG. 13)

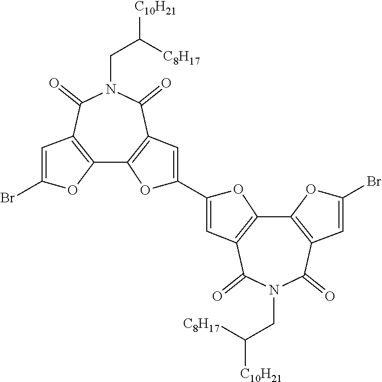

Bromine (0.11 mL, 1.98 mmol) and a catalytic amount of FeCl₃ (2 mg, 2 mol %) were added to a solution of imide L-2BFI (FIG. 13, 0.64 g, 0.66 mmol) in dry CH₂Cl₂ (40 mL) and the reaction mixture was stirred in the dark for 6 hr. After completion of the reaction, it was quenched using a sat. aq. solution of Na₂S₂O₃ and stirred for a further 30 min. The reaction mixture was poured into CH₂Cl₂ (60 mL) and washed with water (2×40 mL), brine, dried (Na₂SO₄), filtered, and concentrated. The residue obtained was purified by flash column chromatography on silica gel using hexane and CH₂Cl₂ (1:1) as eluent to give 3 (FIG. 13) as a light yellow solid (0.66 g, 89%). ¹H NMR (500 MHz, Chloroform-d) δ 7.50 (s, 2H), 7.10 (s, 2H), 4.23 (d, J=7.2 Hz, 4H), 1.89-1.84 (m, 2H), 1.38-1.18 (m 64H), 0.86 (t, J=7.0 Hz, 6H), 0.85 (t, J=7.0 Hz, 6H); ¹³C NMR (126 MHz, Chloroform-d) δ 159.82, 159.39, 144.89, 143.58, 142.16, 126.53, 122.89, 121.80, 116.14, 111.92, 48.74, 36.39, 32.07, 32.06, 31.81, 30.19, 29.81, 29.80, 29.76, 29.71, 29.50, 29.47, 26.60, 22.83, 14.26. HRMS (MALDI) calcd for C₆₀H₈₆Br₂N₂O₈ 1122.473, found 1122.505 (M)⁺.

### L-4BFI (FIG. 13)

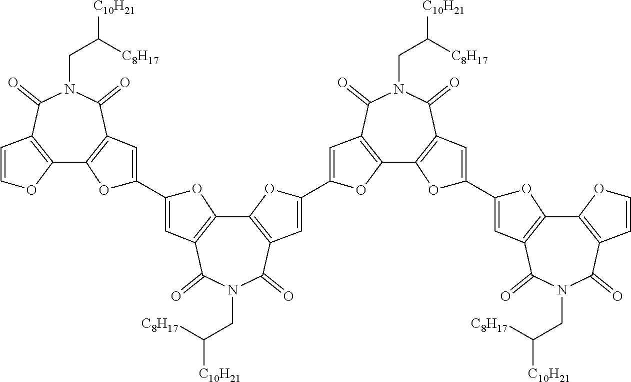

Tetrakis(triphenylphosphine)palladium(0) [(Pd(PPh₃)₄] 25 mg, 0.021 mmol) was added to a solution of 2 (FIG. 13, 0.27 g, 0.42 mmol) and N-2-octyldodecyl-5-(tributylstannyl)-2,2′-bifuran-3,3′-dicarboximide (1) (0.75 g, 0.97 mmol) in dry and degassed toluene (15 mL), and the reaction mixture was stirred at 90° C. for 24 h under nitrogen. Then reaction mixture was cooled to room temperature and the solvent was evaporated under reduced pressure. The residue obtained was purified by flash column chromatography on silica gel using hexane and dichloromethane (1:1 to 9:1) as eluent to give L-4BFI (FIG. 13) as a red solid (0.48 g, 79%). ¹H NMR (500 MHz, Chloroform-d) δ 7.67 (d, J=1.9 Hz, 2H), 7.60 (s, 2H), 7.54 (s, 2H), 7.48 (s, 2H), 7.19 (d, J=1.8 Hz, 2H), 4.26 (d, J=7.2 Hz, 4H), 4.24 (d, J=7.2 Hz, 4H), 1.90 (h, J=6.3 Hz, 4H), 1.44-1.16 (m, 128H), 0.92-0.79 (m, 24H); ¹³C NMR (126 MHz, Chloroform-d) δ 160.29, 159.92, 159.68, 159.62, 145.29, 145.13, 144.40, 144.31, 143.25, 142.35, 142.28, 142.00, 122.85, 122.71, 121.79, 121.55, 114.69, 112.27, 112.20, 111.82, 48.77, 48.63, 36.48, 32.07, 31.90, 31.88, 30.24, 29.85, 29.83, 29.81, 29.80, 29.78, 29.75, 29.73, 29.50, 29.48, 26.67, 22.82, 14.25; HRMS (MALDI) calcd for C₁₂₀H₁₇₄N₄O₁₆ 1927.292, found 1927.291 (M)⁺.

### Compound 3 (FIG. 13)

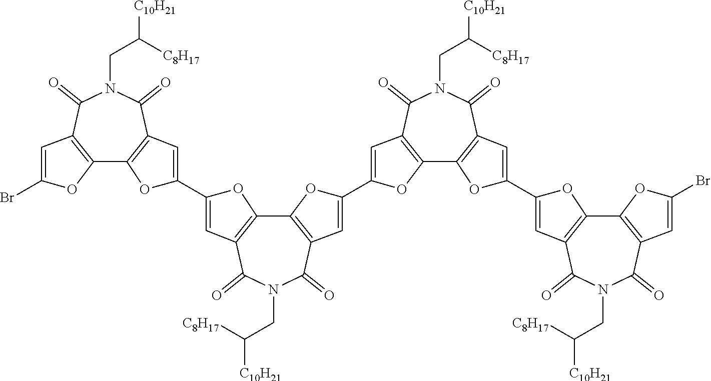

Bromine (0.041 mL, 0.794 mmol) and a catalytic amount of FeCl₃ (0.86 mg, 2 mol %) were added to a solution of imide L-4BFI (FIG. 13, 0.51 g, 0.265 mmol) in CH₂Cl₂ (50 mL) and the reaction mixture was stirred in the dark for 6 h. After completion of the reaction, it was quenched using a saturated solution of Na₂S₂O₃ and stirred for additional 30 min. The reaction mixture was then poured into CH₂Cl₂ (60 mL) and washed with water (2×40 mL), brine, dried (Na₂SO₄), filtered, and concentrated. The residue obtained was purified by flash column chromatography on silica gel using hexane and CH₂Cl₂ (1:1) as eluent to give 3 (FIG. 13) as a light orange solid (0.442 g, 80%). ¹H NMR (500 MHz, Chloroform-d) δ 7.59 (s, 2H), 7.53 (s, 2H), 7.49 (s, 2H), 7.08 (s, 2H), 4.26 (d, J=7.3 Hz, 4H), 4.21 (d, J=7.3 Hz, 4H), 1.92-1.83 (m, 4H), 1.38-1.21 (m, 128H), 0.87-0.83 (m, 24H); ¹³C NMR (126 MHz, Chloroform-d) δ 159.74, 159.69 (2C), 159.35, 145.21, 145.18, 144.77, 143.53, 142.34, 142.25, 142.20, 126.61, 122.95, 122.88, 122.86, 121.86, 116.17, 112.41, 112.24, 112.22, 48.77, 36.48, 36.43, 32.08, 31.89, 31.85, 30.22, 29.84, 29.81, 29.80, 29.78, 29.75, 29.73, 29.53, 29.51, 29.49, 26.64, 22.83, 14.26; HRMS (MALDI) calcd for C₁₂₀H₇₂Br₂N₄O₁₆ 2085.114, found 2085.132 (M)⁺.

### C-4BFI (FIG. 13)

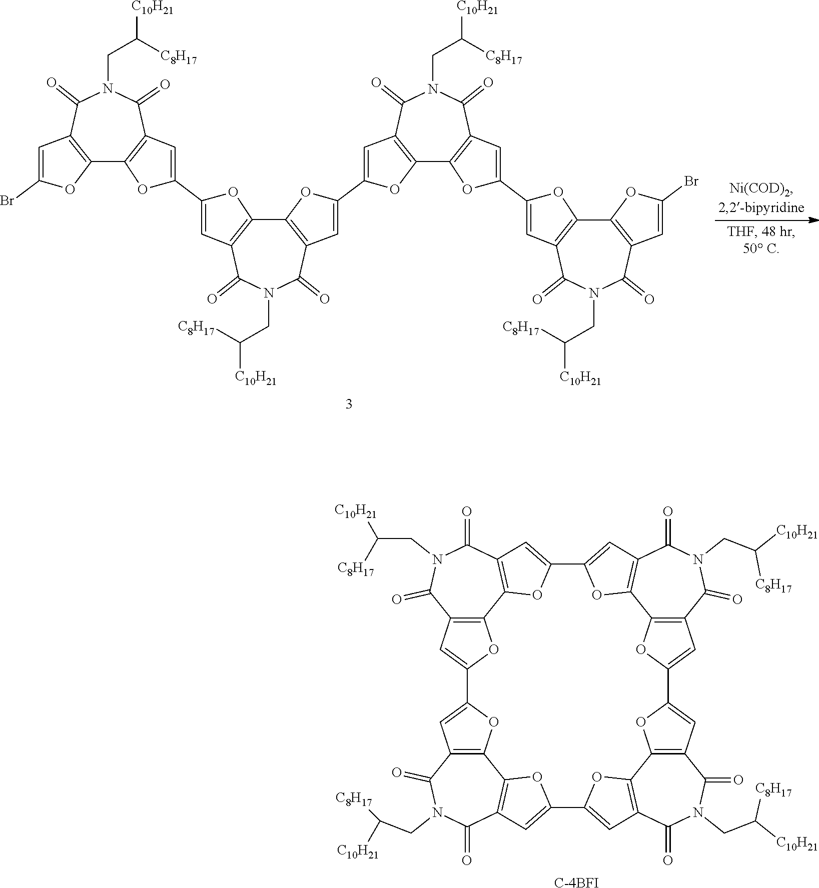

A solution of Bis(1,5-cyclooctadiene)nickel(0) [Ni(COD)₂] (37 mg, 0.13 mmol) and 2,2′-dipyridyl (21 mg, 0.13 mmol) in dry THF (15 mL) was stirred for 15 min at 50° C. under argon atmosphere. After cooling to room temperature, the violet solution was transferred to a solution of compound 3 (FIG. 13, 250 mg, 0.12 mmol) in THF (235 mL). The reaction mixture was stirred for 48 hr at 50° C. under argon atmosphere. The solvent was evaporated under vacuum and the crude product was purified by flash column chromatography on silica gel using hexane and CH₂Cl₂ (1:1) as eluent to give C-4BFI (FIG. 13, as a red solid (120 mg, 52%). ¹H NMR (500 MHz, Chloroform-d) δ 7.30 (s, 8H), 4.19 (d, J=7.2 Hz, 16H), 1.86 (p, J=5.9 Hz, 8H), 1.38-0.99 (m, 128H), 0.87 (t, J=7.0 Hz, 24H); ¹³C NMR (126 MHz, Chloroform-d) δ 159.47, 144.98, 142.54, 123.28, 113.56, 48.86, 36.48, 32.09, 32.07, 31.83, 30.25, 29.85, 29.83, 29.81, 29.76, 29.53, 29.50, 26.63, 22.85, 22.84, 14.27; HRMS (MALDI) calcd for C₁₂₀H₁₇₂N₄O₁₆ 1925.276, found 1925.267 (M)⁺.

### Discussion

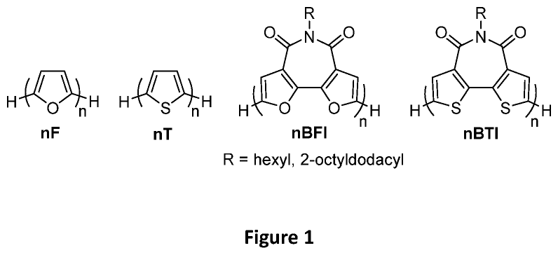

Herein the synthesis of a BFI unit, and its corresponding oligomers and polymers (nBFI) bearing hexyl (nBFI-H) or 2-ocyldodecyl (nBFI-OD) substituents, as new building units (FIG. 1) are presented. It is demonstrated that bifuran-imide shows significantly better stability compared with α-oligofurans, good solid-state packing, as well as good solubility at room temperature, which is important for their processability. These properties allowed the production of high molecular weight polymer, poly(BFI-OD), which was also found to be soluble in a wide range of organic solvents. Unlike its thiophene analogue, the HOMO of poly(BFI-OD), which was obtained by both DFT calculations and from electrochemical measurements, renders these materials p-type semiconductors. The BFI-H unit shows considerable photostability, with little bleaching even after prolonged ambient light exposure. Overall, this technology establishes BFI as a stable furan building unit for organic electronics.

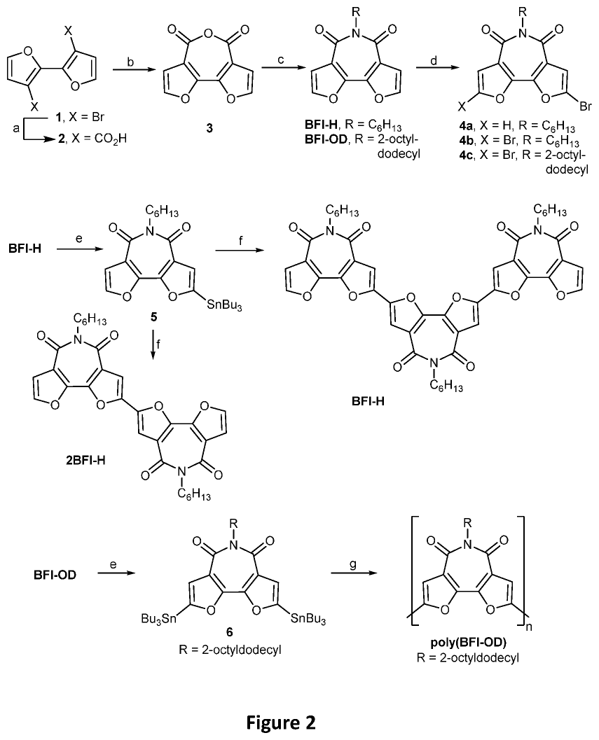

The synthesis of BFI monomer, dimer, trimer, and polymer is depicted in FIG. 2. In the first step, dibromobifuran (1) was converted to bifuran-dicarboxylic acid 2 by lithiation with n-butyllithium at −70° C. and treatment with dry ice. Next, bifuran-dicarboxylic anhydride 3 was obtained in good yield (81%) from 2 by condensation/cyclization in refluxing acetic anhydride. The key building units, bifuran-imides BFI-H (88%) and BFI-OD (79%), were synthesized from anhydride 3 by reaction with hexylamine or 2-octyldodecylamine, respectively, followed by in situ treatment with SOCl₂. Monobromination of BFI-H with molecular bromine (1.2 equiv) gave monobromobifuran-imide 4a with moderate yield (56%) along with dibromobifuran-imide 4b (15%). An alternative high-yield route to the dibromo BFI products 4b (97%) and octyldodecyl)-dibromo-bifuran-dicarboximide 4c (96%) involved the corresponding BFI-H or BFI-OD, respectively, under similar conditions with excess Br₂. To synthesize 2BFI-H and 3BFI-H, the Stille coupling reagent N-hexyl-5-(tributylstannyl)-2,2′-bifuran-3,3′-dicarboximide (5) was obtained in moderate yield (52%) from BFI-H by lithiation with lithium diisopropylamide (LDA) at −85° C. followed by treatment with tributyltin chloride. Next, the Pd-catalyzed Stille coupling of 5 with monobromobifuran-imide (4a) and dibromobifuran-imide (4b) delivered the dimer 2BFI-H (61%) and trimer 3BFI-H (45%), respectively.

Attempts to synthesize the homopolymer of BFI-H by Stille polymerization of 4b resulted in an insoluble polymer. The distannylated product of BFI-OD, namely 6, was therefore coupled with 4c to produce poly(BFI-OD), which is considerably more soluble than the hexyl analogue. This was first evident from the NMR spectrum of poly(BFI-OD), which could be recorded in CDCl₃ with no heating, whereas the NMR spectrum of the corresponding thiophene-based homopolymer, poly(BTI-OD) was recorded at 120° C. in 1,2-tetrachloroethane. Gel permeation chromatography (GPC) was used to determine polymer molecular weight versus polystyrene. Poly(BFI-OD) was found to have a molecular weight (Mw) of 27000 D and a polydispersity index (PDI) of 2.1, corresponding to a mean chain length of ˜60 monomer units (˜120 furan rings). This is significantly longer than for the thiophene analogue, which consists of only ˜15 monomer units. This difference in mean chain length reflects the better solubility of poly(BFI-OD), as greater solubility permits the formation of longer polymer chains.

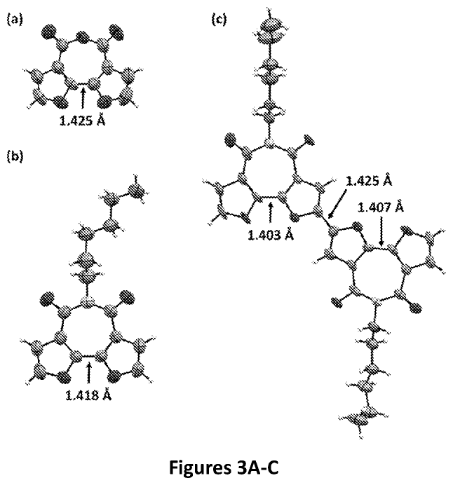

The single X-ray crystal structures of 3, BFI-H, and 2BFI-H were obtained by slow diffusion of hexane into dichloromethane at room temperature. The anhydride 3 and BFI-H (FIGS. 3A-B) have conjugated backbones and planar conformations with the C—C bond length between two furan rings measured at 1.425 Å and 1.418 Å, respectively. These distances are shorter than those for bridged bifurans with phosphor atoms (1.430 Å). The dimer 2BFI-H displays shorter C—C bond lengths between the two furans in the same imide unit (1.403 Å and 1.407 Å) and interunit bond length of 1.425 Å, which is an indication of the strong quinoid character of this π-conjugated backbone. The backbone of 2BFI-H is planar, which is an additional indication of quinoid character, and a hallmark of furan oligomers. 2BFI-H shows π-π interactions in a slightly stagged arrangement with an interplanar distance of 3.25 Å.

One of the most pronounced differences between furan and thiophene is the exo ring angle, with a value of 129° for 2,5-dimethylthiophene and 134° for 2,5-dimethylfuran. Consequently, bifurans fused to a 5 membered ring are expected to be highly strained, whereas fusion to a 7 membered ring should produce significantly less strain.

UV-vis absorption and emission spectra of BFI-H, 2BFI-H, 3BFI-H, BFI-OD, and poly(BTI-OD) in chloroform are displayed in FIGS. 5A-B together with the thiophene analogue, BTI-OD, for comparison. It is interesting to note that, whereas all the furan-containing analogues display clear vibronic shoulders in the absorption spectra, BTI-OD is featureless, indicating a more flexible backbone, even for the fused system. An additional indication of the greater rigidity of the furan-containing analogues is their lower Stokes shift (0.52 eV vs. 0.66 eV for BFI-OD and BTI-OD, respectively). The dimer and trimer display a smaller Stokes shift of 0.26 eV for both (Table 1). The polymer, poly(BFI-OD), also displays a small Stokes shift (0.02 eV) with λabs=532 nm and λem=537 nm, indicating a highly rigid backbone, despite the high solubility (Table 1). No solvatochromic shift was observed for the absorption spectra upon changing the solvent polarity, from hexane to acetonitrile.

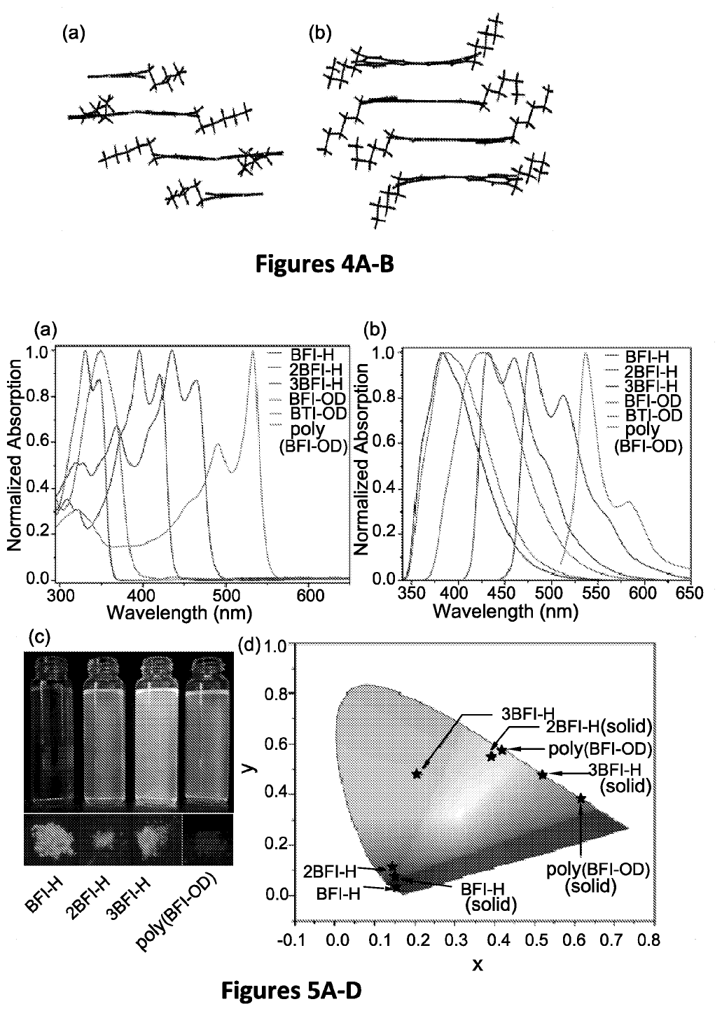

For 2BFI-H and 3BFI-H, the emission spectra (FIG. 5B) show maxima at 432 nm and 472 nm with shoulder peaks at 459 and 513 nm, respectively. The quantum efficiencies (φf) range between 0.65 and 0.81 for all oligomers (Table 1). It is interesting to note that the quantum efficiency remains high also for the polymer poly(BFI-OD), with φf=0.71 (Table 1). FIG. 5C shows the fluorescence images of BFI-H, 2BFI-H, 3BFI-H, and poly(BFI-OD) in solution (CHCl₃) and in the solid state at 365 nm. FIG. 5D places BFI-H, 2BFI-H, 3BFI-H, and poly(BFI-OD) on the ICE 1931 chromaticity diagram in solution (CHCl₃) and in the solid state. BFI-H shows relatively high solid-state quantum efficiency with φf=0.18, as well as strong π-π interactions (FIG. 4B), and the combination of these factors with CIE coordinates of (0.15, 0.07) renders it a good candidate for blue light emitting devices, with CIE coordinates very close to those of the European Broadcasting Union (EBU) blue standard (0.15, 0.06).

To attain a better understanding of the strong fluorescence properties of BFI-containing materials, their fluorescence lifetime (K_{f}) was measured. The K_{NR} values (Table 1) for the monomers are very small, in the range of 0.05-0.06 ns⁻¹, indicating a rigid backbone, and increase for the dimer and trimer. However, the polymer has a similarly low value as the trimer, of 0.18 ns⁻¹.

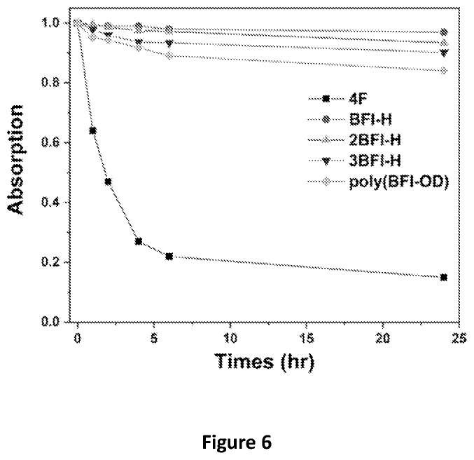

As oligofurans are prone to photooxidation, the photostability of the BFI series was studied by exposing them to identical ambient light conditions, together with 4F for comparison. As expected, 4F underwent rapid photooxidation, with less than 50% of the initial absorption remaining after 2 h (FIG. 6). In contrast, the BFI-H unit showed almost no bleaching, even after prolonged exposure of 24 h. It is important to note that both 2BFI-H and 4F having similar λabs values and extinction coefficients. Longer oligomers show more significant bleaching, but are considerably more stable than 4F, with less than 10% bleaching after 24 h for poly(BFI-OD). Previous efforts to obtain polyfurans always resulted in materials which underwent bleaching in minutes. To the best of the inventors' knowledge, poly(BFI-OD) a polyfuran derivative stable under ambient conditions.

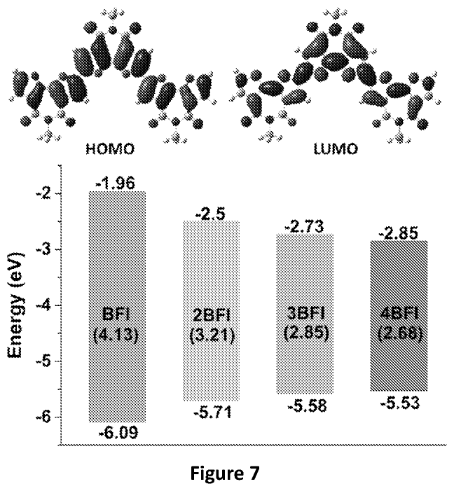

In order to obtain a better assessment of the energy levels of BFI-containing materials, their properties were studied using DFT calculations at the B3LYP/6-31G(d) level of theory. FIG. 7 displays the HOMO and LUMO levels of nBFI (n=1-4). Unlike the corresponding BTI, which can be considered n-type materials, the HOMO level remains high for BFI, with 3BFI having similar HOMO and LUMO levels as for sexithiophene (6T) which is considered a p-type semiconductor with ambipolar properties. It is therefore reasonable to assume that 3BFI and longer bifuran-imides have the potential to function as ambipolar semiconductors in OFET devices.

TABLE 1
Photophysical data of bifuran-imide-based compounds

| | | | Stokes | | | | | | | | | |
|---|---|---|---|---|---|---|---|---|---|---|---|---|
| | λ_{abs}^{a} | λₑₘ^{a} | shift | ε^{a} | | Φ_{f} | τ_{F}^{a} | k_{F}^{c} | k_{NR}^{d} | E_{g (opt)}^{e} | HOMO | LUMO |
| Comp. | (nm) | (nm) | (eV) | (M⁻¹.cm⁻¹) | Φ_{f}^{a} | (solid) | (ns) | (ns⁻¹) | (ns⁻¹) | [eV] | [eV] | [eV] |
| BFI-H | 330, 343 | 383 | 0.52 | 13714 | 0.80 | 0.18 | 4.14 | 0.19 | 0.05 | 3.44 | −6.05 | −2.61 |
| 2BFI-H | 396, 415 | 432, 459 | 0.26 | 37353 | 0.81 | 0.06 | 1.74 | 0.46 | 0.11 | 2.85 | −5.71 | −2.86 |
| 3BFI-H | 365, 435, | 478, 513 | 0.26 | 57600 | 0.65 | 0.06 | 1.78 | 0.36 | 0.20 | 2.57 | −5.69 | −3.12 |
| | 457 | | | | | | | | | | | |
| BFI-OD | 331, 344 | 385 | 0.52 | 10318 | 0.77 | — | 4.08 | 0.19 | 0.06 | 3.44 | −5.94 | −2.5 |
| BTI-OD | 348 | 427 | 0.66 | 11670 | 0.78 | — | 5.83 | 0.13 | 0.04 | 3.22 | −6.03 | −2.81 |
| Poly(BFI-OD) | 492, 532 | 537, 584 | 0.02 | — | 0.71 | 0.05 | 1.61 | 0.44 | 0.18 | 2.26 | −6.30 | −4.04 |
| | | | | | | (0.03)^{b} | | | | | | |

^{a}Measured in the CHCl₃;
^{b}measured as thin film;
^{c}Calculated according to the equation k_{F} = Φ_{f} /τ_{F};
^{d}Calculated according to the equation Φ_{f} = k_{F}/(k_{F} + k_{NR});
^{e}E_{gap} =1242/λₒₙₛₑₜ; E_{HOMO} = −(4.8 −E_{Fc/Fc+}^{1/2} + Eₒₙₛₑₜ^{oxi1}); E_{LUMO} = E_{HOMO} + E_{gap}.

The synthesis, structure, and properties of the first macrocycle having an oligofuran backbone, the bifuranimide α,α′-tetramer C-4BFI, shown below, has also been achieved (each R may be the same or different and may be as selected from H, C₁-C₃₀alkyl, C₂-C₁₀alkenyl, C₂-C₁₀alkynyl, C₆-C₁₀aryl, C₅-C₁₀heteroaryl comprising between 1 and 5 heteroatoms selected from N, O, S; —CN and —C₂H).

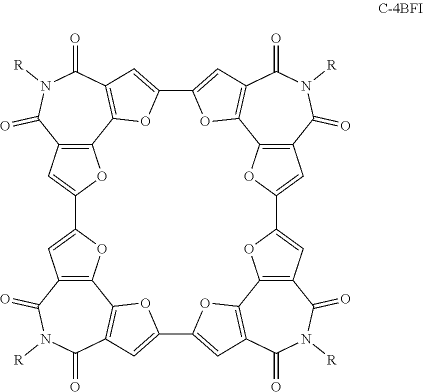

In accordance with the inventors' calculations, and in contrast with macrocyclic oligothiophenes of the same size, C-4BFI is planar, as verified by crystallographic analysis, and exhibits remarkably strong intermolecular interactions. In solution and as a solid, C-4BFI self-assembles via π-stacking. Scanning tunneling microscopy (STM) imaging reveals the formation of ordered multilayers at the solid-liquid interface.

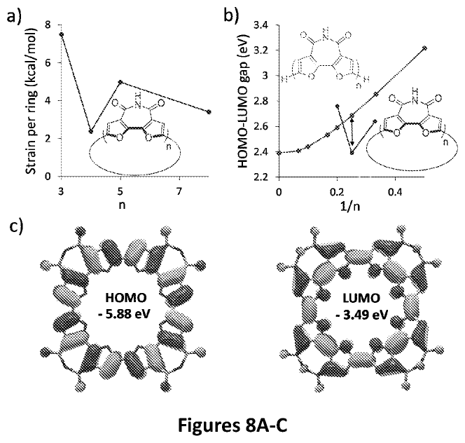

To find the ideal candidate for macrocyclization, the inventors calculated the electronic structures (at the DFT/B3LYP/6-311G(d) level) of C-nBFI molecules of various sizes (FIG. 8A). It was found that the tetramer, C-4BFI, has the lowest strain energy (2.4 kcal/mol per bifuran unit), being lower than that of the smaller C-3BFI (7.5 kcal/mol) and of the larger C-5BFI (5.0 kcal/mol). In addition, the backbone of C-4BFI is predicted to be planar which maximizes the π-conjugation. Calculations predicted that the HOMO-LUMO gap for C-4BFI (2.38 eV) is 0.3 eV lower than that of the linear tetramer (L-4BFI), and is almost identical to the bandgap of the linear polymer (FIG. 8B). The predicted HOMO-LUMO gap in C-3BFI is also smaller compared with the linear analogue L-3BFI (by 0.2 eV) but the C-5BFI macrocycle has ˜0.2 eV larger gap than L-5BFI, which is attributed to its non-planarity. Both the HOMO and LUMO resemble those of the parent macrocyclic furans, with a small contribution from the imide group to the LUMO (FIG. 8C). The computational results thus reveal C-4BFI as an optimum target for cyclization.

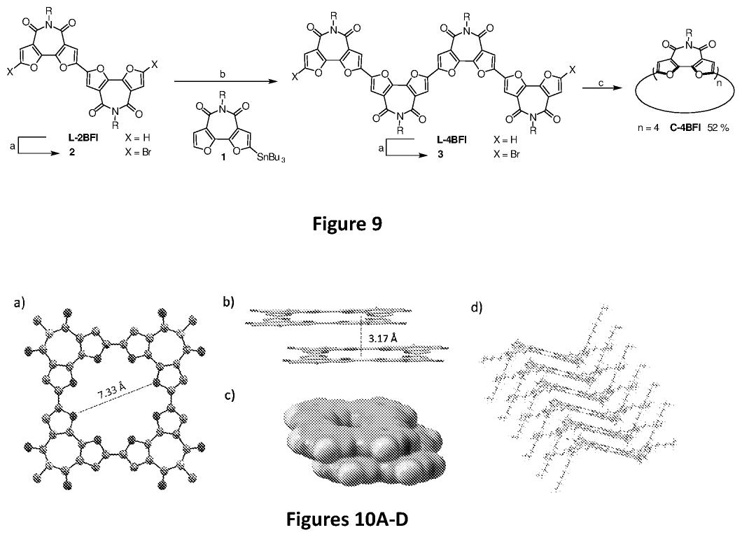

The synthesis started with preparation of the linear tetramer L-4BFI by Stille coupling of stannane 1 with the dibrominated product of L-2BFI (2) (FIG. 9). The 2-octyldodecyl side chains were introduced for solubility (attempts to use n-hexyl side chain resulted in insoluble linear tetramer). The coupling product, L-4BFI, was then brominated using Br₂ in the presence of FeCl₃ to yield 3. Finally, macrocyclization was performed by adding a stoichiometric amount of Ni(COD)₂ and 2,2′-dipyridy to a dilute solution (5×10⁻⁴ M) of 3 at 50° C. to yield C-4BFI (52%) after 48 h. The macrocycle was characterized using NMR and by MALDI-TOF.

Single crystals of C-4BFI were grown by slow diffusion of acetone into dichloromethane solution, yielding red needles. The single crystal X-ray analysis shows that the macrocycle is planar (FIG. 10A).

This contrasts sharply with both calculated and experimental data for oligocyclicthiphenes. Calculations show that cyclic octathiophene adopts a non-planar cone conformation. Likewise, in the smallest structure available for macrocyclic thiophenes, the thiophene rings twist with dihedral angles of 26-34°. The bond distance between the adjacent BFI units is 1.43 Å, which is an indication of strong conjugation and quinoid character. The inner diameter of C-4BFI macrocycles is 7.33 Å, corresponding to a van der Waals cavity of 4.3 Å. This is similar to the predicted inner cavity for 24-crown-8 ether (4-4.5 Å),38 yet achieved in a highly rigid π-conjugated structure that is interesting as a host for supramolecular engineering of optoelectronic materials.

The macrocycles pack in a slip-stacked motif (FIG. 10B), with a very short intermolecular π-π stacking distance of 3.17 Å (closest C . . . C distance 3.26 Å). The short packing distances contrast sharply with those found in thiophene-acetylene macrocycles (3.7 Å) or in C-10T (>8 Å), and is even closer than the interplane distance of graphite (3.35 Å). This tight packing is a result of the planarity of the macrocycle and the quadrupole moment brought about by four imide groups, which induce strong 7-71 interactions. The calculated electrostatic potential (ESP) map of the dimer, extracted from the X-ray structure, explains the slippage as resulting from interactions between the electron-poor imide groups and the electron-rich bifuran units (FIG. 10C). The large 2-octyldodecyl groups protrude up and down on four sides of the macrocycle, forming isolated π-channels with strong but one-dimensional interactions (FIG. 10D).

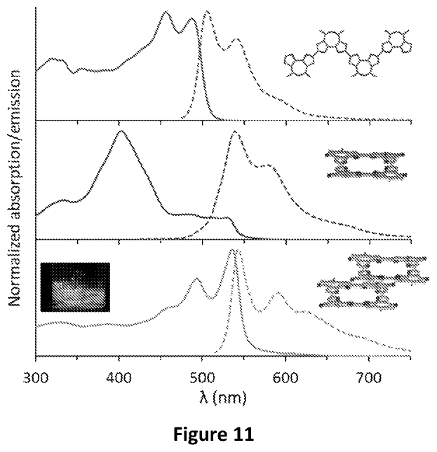

The UV-Vis absorption spectrum of the linear tetramer, L-4BFI, displays the expected π-π* transition, with two vibronic peaks at 457 nm and 489 nm corresponding to a C═C backbone stretch of 0.17 eV (˜1400 cm−1; FIG. 11). The emission spectrum shows the similarly structured S1→S0 transition, with a Stokes shift of 0.09 eV. For the macrocyclic oligofuran C-4BFI, the absorption maximum is at 401 nm (FIG. 3, blue trace), corresponding to the S0→S2 transition (extinction coefficient of 1.1×10⁵ cm⁻¹M⁻¹). The shoulder that appears in the ˜480-520 nm range is likely associated with the S0→S1 transition, which is Laporte forbidden due to a conservation of orbital symmetry in the centrosymmetric macrocycle. The emission spectrum shows a similar pattern for the macrocycle and the linear tetramer, corresponding to the S1→S0 transition. However, the emission maximum is bathochromically shifted by ˜0.2 eV for C-4BFI compared with L-4BFI, which is in-line with the calculated difference in their HOMO-LUMO gap. The fluorescence quantum yield for the macrocycle measured in chloroform at 450 nm excitation is 18%, which is smaller than that of the linear oligomer (61%), in line with the symmetry-forbidden S1→S0 transition. However, it is larger than the fluorescence quantum yield of macrocyclic oligothiophenes.

The absorption spectrum of C-4BFI in hexane at high dilution is similar to that observed in dichloromethane, with a strong S0→S2 transition and a weak S0→S1 tail. Increasing the concentration leads to the emergence of new transitions at 535 nm and 489 nm, until a solid film is formed in which only these new transitions can be observed (FIG. 11). The emission spectrum is nearly a mirror image of the absorption, with an extremely small Stokes shift of ˜0.03 eV, indication of a rigid structure. Aggregation is also easily observed in hexane solutions by dynamic light scattering measurements (DLS). At a concentration of 10⁻⁵ M, C-4BFI forms aggregates with a hydrodynamic diameter ˜45 nm; these grow to large particles (˜90 nm) as the concentration increases to 10⁻⁴ M. With time, these aggregates form a red film of the container walls. Atomic Force Microscopy (AFM) imaging of this film shows particles up to ˜100 nm size, in line with DLS measurements, and reveals their crystalline nature. By comparison, the linear tetramer L-4BFI shows a much lower tendency to aggregate, with no observable aggregates at a concentration of 10⁻⁵ M and only small (5 nm) aggregates appearing at 10⁻⁴ M. 1H-NMR shows the concentration dependence of the chemical shift of the β-proton up from 10-5 M, which supports strong intermolecular interactions. Samples dissolved in toluene display higher solubility compared with hexane, and no change in spectra even at 10⁻³ M concentration, supporting that π-π interactions of the macrocyclic core is the main cause for the observed aggregation.

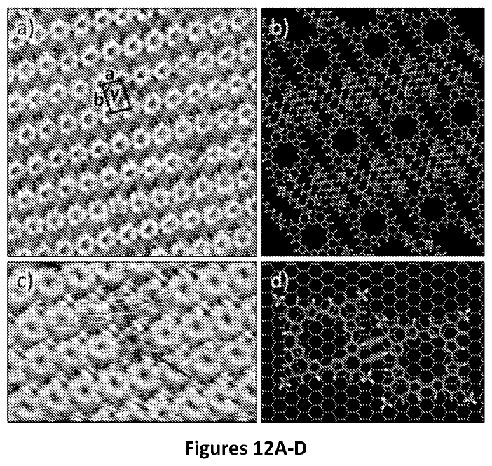

The self-assembly of C-4BFI at the solid-liquid interface was imaged using STM. The macrocycles adopt a lamellar arrangement with an oblique unit cell (a=1.7±0.1 nm, b=2.4±0.1 nm, γ=84±1°, FIG. 12A). The conjugated macrocyclic core appears bright and the dark region between the macrocycle rows corresponds to interdigitated alkyl chains. The central cavities of the macrocycles are clearly resolved. Molecular modelling of the observed pattern suggests that, within the rows, the macrocycles are linked by two-point hydrogen bonding involving furan CH donors and carbonyl acceptors (FIG. 12B-,D). Such assembly allows for interactions between only half of the alkyl chains (4 out of 8) and the HOPG surface. Presumably, the other alkyl chains are adsorbed on top of themselves (FIG. 12B) as has been observed for other alkylated molecule. It is to note the presence of a pronounced tendency of the macrocycle to form multilayers at the liquid-solid interface, in line with its aggregation in solution. FIG. 12C shows the multilayer, with molecules missing from the upper layer giving the appearance of holes in which a lower contrast molecule (of the lower layer) can still be identified.

### CONCLUSIONS

In summary, a new building unit, bifuran-imide, was introduced and used to synthesize oligomers and the homopolymer. Unlike the corresponding thiophene analogue, the polymer was highly soluble, allowing us to obtain a polymer with high molecular weight. Notwithstanding their solubility, these materials have a strong quinoid character, as evident from the short interring bond in the X-ray structure, and the vibronic features in their absorption spectra. The obtained oligomers and polymers exhibit strong fluorescence with quantum yields reaching up to 81% in solution. The monomer, BFI-H, also demonstrated strong blue emission in the solid state. Compared with their parent oligofurans, bifuran-imides are significantly more stable under ambient conditions. It is therefore suggested that this new unit can replace furan in organic electronic materials.

1. A compound of the general Formula (III): 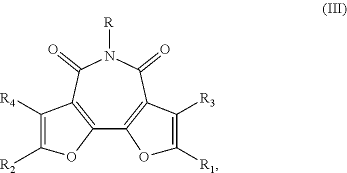
wherein
R is selected from H, C₁-C₃₀alkyl, C₂-C₁₀alkenyl, C₂-C₁₀alkynyl, C₆-C₁₀aryl, C₅-C₁₀heteroaryl comprising between 1 and 5 heteroatoms selected from N, O, S; —CN and —CO₂H;
each of R₁, R₂, R₃ and R₄, independently of the other, is selected from hydrogen, C₁-C₃₀alkyl, C₂-C₁₀alkenyl, C₂-C₁₀alkynyl, C₆-C₁₀aryl, C₅-C₁₀heteroaryl comprising between 1 and 5 heteroatoms selected from N, O, S; —CN, —CO₂H, —OH, —SH, —NR′R″R″′, —NO₂, (C₁-C₃₀alkyl)₃Si—, (C₁-C₃₀alkyl)₃Sn—, trifluoromethanesulfonate (triflate), halogen and R_{A}, wherein each of R′, R″ and R″′ is the same or different and is selected from hydrogen, C₁-C₁₀alkyl, C₂-C₁₀alkenyl, C₂-C₁₀alkynyl, C₆-C₁₀aryl; wherein in case of charged amine groups, the group is associated with at least one counter-ion.

2. The compound according to claim 1, wherein at least one of R₁ and R₂ is H.

3. The compound according to claim 1, wherein at least one of R₁ and R₂ is a halide selected from Br, I, Cl, and F.

4-8. (canceled)

9. The compound according to claim 1, wherein RA is a group selected from: 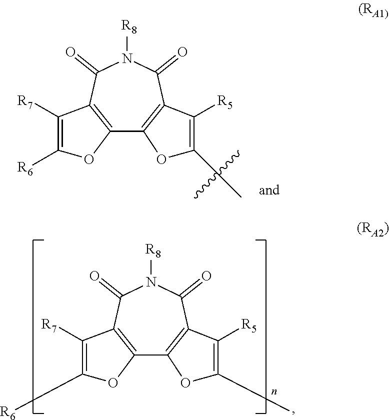
wherein
the wavy line designates a bond formed between R_{A1} and position R₁, R₂, R₃ or R₄ on a compound of Formula (III);
each of R₅, R₆, R₇ and R₈, independently of the other, is selected from hydrogen, C₁-C₃₀alkyl, C₂-C₁₀alkenyl, C₂-C₁₀alkynyl, C₆-C₁₀aryl, C₅-C₁₀heteroaryl comprising between 1 and 5 heteroatoms selected from N, O, S; —CN, —CO₂H, —OH, —SH, —NR′R″R″′, —NO₂, (C₁-C₃₀alkyl)₃Si—, (C₁-C₃₀alkyl)₃Sn—, trifluoromethanesulfonate (triflate) and halogen, wherein each of R′, R″ and R″′ is the same or different and is selected from hydrogen, C₁-C₁₀alkyl, C₂-C₁₀alkenyl, C₂-C₁₀alkynyl, C₆-C₁₀aryl; wherein in case of charged amine groups, the group is associated with at least one counter-ion; or a covalent bond;
n is an integer indicating the number of repeating monomers, being between 1 and 100;
such that when R_{A} is R_{A2}, in a repeating end of chain monomer, R₆ may be H or C₁-C₃₀alkyl; and
in a repeating mid-chain monomer, R₆ is a bond connecting two monomers.

10-13. (canceled)

14. The compound according to claim 9, wherein R₆ is Br or (C₁-C₃₀alkyl)₃Sn—.

15. The compound according to claim 9, wherein R₆ (C₁-C₃₀alkyl)₃Sn— and each of R₅, R₇ and R₈ is C₁-C₃₀alkyl or H.

16. (canceled)

17. A dimer, a trimer, an oligomer or a polymer of a compound according to claim 1.

18. (canceled)

19. A compound of the general Formulae (IV) or (V) or (VI): 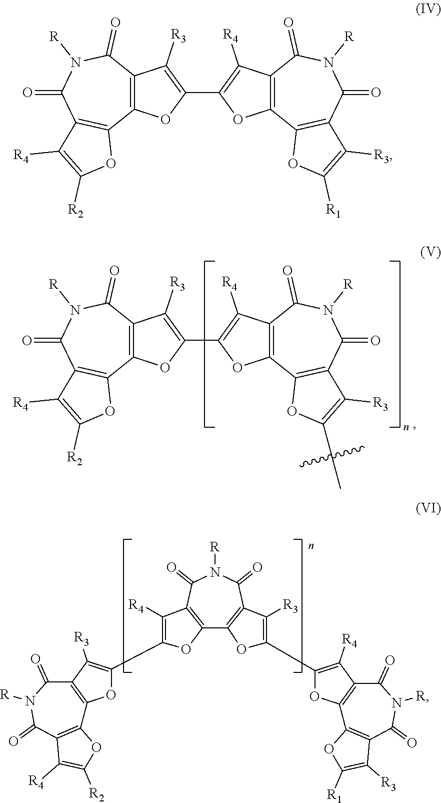
wherein
in each of compounds of Formulae (IV), (V) and (VI): each R, R₁, R₂, R₃, R₄, R is the same or different and selected as recited in claim 1.

20. The compound according to claim 19, wherein in a compound of Formula (V), the wavy line designates a point or bond of connectivity and n is the number of monomer units, being selected between 1 and 30.

21. The compound according to claim 1, having the structure: 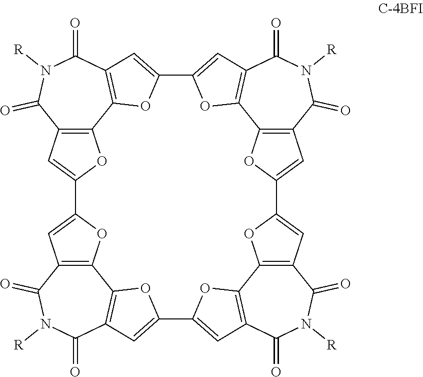
wherein each R, being the same or different, is as selected in claim 1.

22-35. (canceled)

36. A compound of general Formula (II): 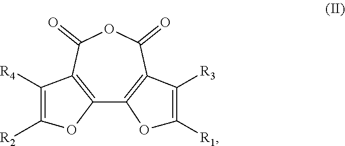
wherein
each of R₁, R₂, R₃ and R₄, independently of the other, is selected from hydrogen, C₁-C₃₀alkyl, C₂-C₁₀alkenyl, C₂-C₁₀alkynyl, C₆-C₁₀aryl, C₅-C₁₀heteroaryl comprising between 1 and 5 heteroatoms selected from N, O, S; —CN, —CO₂H, —OH, —SH, —NR′R″R″′, —NO₂, (C₁-C₃₀alkyl)₃Si—, (C₁-C₃₀alkyl)₃Sn—, trifluoromethanesulfonate (triflate) and halogen, wherein each of R′, R″ and R″′ is the same or different and is selected from hydrogen, C₁-C₁₀alkyl, C₂-C₁₀alkenyl, C₂-C₁₀alkynyl, C₆-C₁₀aryl; wherein in case of charged amine groups, the group is associated with at least one counter-ion.

37-43. (canceled)

44. A method of synthesis of a dimer, trimer, an oligomer or a polymer of a compound of general Formula (III), according to claim 1, the method comprising reacting a functionally substituted bifuran-imide under coupling conditions, to afford a dimer of the bifuran imide.

45-53. (canceled)

54. A blue emitting device comprising a compound according to claim 1.

55. A device comprising a blue emitter in the form of a compound according to claim 1.

56. (canceled)
